(19) United States
(12) Patent Application Publication
(10) Pub. No.: US 2026/0014227 A1
(43) Pub. Date: Jan. 15, 2026

(54) DESLORELIN USE IN CHEMICAL CASTRATION OF A NON-HUMAN MAMMAL RELATED TO PK/PD INTERACTION

(71) Applicant: VIRBAC, Carros (FR)

(72) Inventors: Gwenaëlle LE BARS, Le Cannet (FR); Jean-Baptiste RASCLE, Villeneuve-Loubet (FR)

(73) Assignee: VIRBAC, Carros (FR)

(21) Appl. No.: 18/880,070

(22) PCT Filed: Jun. 29, 2023
(86) PCT No.: PCT/EP2023/067889

§ 371 Date: Dec. 30, 2024

Related U.S. Application Data
(60) Provisional application No. 63/357,234, filed on Jun. 30, 2022.

(51) Int. Cl. A61K 38/09; A61P 15/16; A61P 15/18

(52) U.S. Cl. CPC A61K 38/09; A61P 15/16; A61P 15/18

(57) ABSTRACT

Deslorelin, in a medicinal or a non-medicinal product, for its use in chemical castration of a non-human mammal within 1 month after administration, wherein upon administration, the Cmax of deslorelin, at around 1 to 2 hours after administration, exceeds 7000 pg/mL and a deslorelin concentration in the non-human mammal plasma is at least 10 pg/mL. Also, a sustained release drug delivery system for implementing this use. Further, a method for chemically castrating a non-human animal including the steps of (a) administering an effective amount of deslorelin so as to obtain a Cmax concentration between 4000 pg/mL and 40000 pg/ml of plasma of deslorelin or an equivalent amount of a pharmaceutically acceptable salt thereof within first 24 h after injection; and thereafter, (b) administering a maintenance dose so that mean concentration is above 10 pg/ml of plasma of deslorelin or an equivalent amount of a pharmaceutically acceptable salt thereof.

### TECHNICAL FIELD

The present disclosure generally relates to pharmaceutical compositions of deslorelin for use in chemical castration of a non-human mammal, preferentially in a companion animal.

### BACKGROUND

Deslorelin is a gonadotropin analog of gonadotropin-releasing hormone (GnRH) and acts by suppressing gonadopituitary axis function when given in low continuous doses.

GnRH is a hypothalamic decapeptide, which acts at the top of the cascade that coordinates the function of the hypothalamic-pituitary gonadal axis. GnRH will bind to GnRH receptors (7 transmembrane receptors) causing the pituitary to produce and release two key gonadotropins, follicle stimulating hormone (FSH) and luteinizing hormone (LH), which in turn control gonadal function:
LH (Luteinizing Hormone), in addition to its role in gametogenesis, promotes the production of sex steroids (androgens, estrogens and progesterone);
FSH (Follicle Stimulating Hormone) stimulates the production of gametes (spermatozoa and eggs).

When GnRH binds to the receptors, it increases the synthesis of the mRNA of its own receptor and raises the sensitivity of the pituitary to GnRH. A continuous secretion of GnRH has an opposite effect (a collapse of LH and FSH secretion is observed: one can speak of desensitization). (Finch A. R. et al., Agonist-induced internalization and downregulation of gonadotropin-releasing hormone receptors. Am. J. Physiol. Cell Physiol. 2009 September, 297 (3)).

Pituitary desensitization to GnRH is triggered by internalization of GnRH receptors (which are no longer present to bind to the cell surface) and inactivation of the intracellular signaling cascade. Once desensitization of pituitary cells to GnRH is initiated, LH concentrations fall to undetectable levels and thus fail to support testosterone and sperm production.

In order to solve a contraceptive problem in animals, some people had the idea to use GnRH agonists continuously and over a long period of time as a contraceptive. This is how the idea of designing a deslorelin implant that continuously diffuses a GnRH agonist (deslorelin) came about.

This type of implant is used in two different ways:
Fertility induction (short term implant): Ovuplant
Induction of infertility by long-term action: Suprelorin

It is known in the state of the art that these agonist-based implants have two phases of action, during the injection of the implant, there is a release of deslorelin which will induce a strong stimulation of the LH and FSH production cascade, this step is called “flare-up”. In male dogs, this induces a production of LH and FSH leading to a strong synthesis of testosterone. The males are then super fertile (improved sperm quality, improved motility).

After this “flare-up” period, a phase of infertility is established with adverse effects on all aspects of sperm quality and testosterone concentration (Goericke-Pesch S., Long-term effects of GnRH agonists on fertility and behaviour. Reprod. Domest. Anim., 2017, 52 Suppl 2:336-347).

In addition to being tested in dogs, studies have been conducted in cats and pussies (Fontaine C., Long-term contraception in a small implant: A review of Suprelorin (deslorelin) studies in cats., J. Feline Med. Surg., 2015, 17 (9), pages 766-771). This shows the effectiveness of the implant as a contraceptive in both males and females. During the period of fertility suppression, steroid hormone concentrations below 1 ng/ml for progesterone and 10 pg/ml for estradiol were measured in females; in male cats, plasma testosterone concentration remained at basal levels (<0.1 ng/ml). The duration of action with a 4.7 mg implant in males was 78.8±12.9 weeks and ranged from 61.7 weeks (approximately 15 months) to 100.7 weeks (approximately 25 months).

Other studies have been performed in leopards, rats, coyotes . . . .

According to the prior art, deslorelin also has a contraceptive effect in females, although the effect varies depending on the species.

As far as is known in the bitch, deslorelin is an effective contraceptive for a long period of time, but in the short term it causes estrus as an undesirable effect. The contraceptive effect could be followed by progesterone analysis with a concentration above 24 ng/ml during the luteal phase (Brändli Sp et al., Long-term effect of repeated deslorelin acetate treatment in bitches for reproduction control, Theriogenology. 2021 Oct. 1, 173, pages 73-82).

In order to solve the problem of estrus induction, it was tested to implant this implant in prepubertal bitches. The effectiveness of the implant was measured by measuring 17-beta estradiol (E2) and progesterone (P4) with a fluorescent enzyme assay. The implant allowed a decrease of these hormones (basal level at more than 10 ng/ml): P 4 between 0.3 and 1.1 ng/ml (0.6±0.2 ng/ml) and E 2 varied between 8 and 138 pg/ml. Thus deslorelin is known to be an early and effective contraceptive in the prepubertal bitch delaying puberty by at least 4.5 months, but the effect can be maintained with repeated treatments. (G Marino et al., deslorelin implants in pre-pubertal female dogs: short- and long-term effects on the genital tract. Reprod. Domest. Anim., 2014), 49 (2), pages 297-301).

It is also known to have a contraceptive effect in cats, for which they evaluated the ovarian activity by monitoring the E2 (estradiol) concentration in the feces. The implant suppressed estrous behavior and decreased estradiol secretion (mean 128.48 ng/g; control: 283.26 ng/g) in all cats for approximately 668 days (23 months). (T S F Toydemir et al., Effects of the GnRH analogue deslorelin implants on reproduction in female domestic cats. Theriogenology. 2012 February, 77 (3), pages 662-674; and Fontaine C. 2015, cited above).

In ferrets, the 4.7 mg deslorelin implant has also shown a contraceptive effect, the side effect is always the same: estrus. Thus, ferrets showed intense signs of estrus, with vulvar swelling, within four days of implant insertion. Two weeks after insertion, spontaneously. However, fecal progesterone concentrations in these animals remained low throughout the 8.5-month sampling period, indicating that they did not ovulate. The deslorelin implant had an inhibitory effect on ovarian function for 698 (+122) days. (A Proháczik et al., Comparison of four treatments to suppress ovarian activity in ferrets (Mustela putorius furo)., Vet. Rec., 2010 Jan. 16, 166 (3), pages 74-78).

This implant has even been tested in large mammals, tigresses. In fact, the implant has made it possible to reversibly regulate the reproduction of tigresses with the injection in some cases of several implants. The average time to conception was 50.7 months (with 4.7 mg implants) and 51.9 months for 9.4 mg implants (A Guthrie et al., The past, present and future of hormonal contraceptive use in managed captive female tiger populations with a focus on the current use of deslorelin acetate, Zoo Biol., 2021, 40 (4) pages 306-319).

Rationale: Until now, the known data did not show a PK/PD (pharmacokinetic/pharmacodynamic) relationship and the efficacy of the product was explained by the very large burst shortly after implantation and a very long duration of GNRH receptors saturation. The current data in dogs indicate a presence of deslorelin for 2.5 months while the effect is observed for at least 6 months (source:
EMA website: https://www.ema.europa.eu/en/documents/product-information/suprelorin-epar-product-information_en.pdf).

Today, new data have been generated and are showing that deslorelin can be measured as long as the effect is observed in dogs and cats males and females.

Accordingly, it is now demonstrated that in a non-human animal not previously chemically castrated, the ‘burst’ (also named herein after or “flare up”) effect is necessary in order to induce the castration after a period of super fertility. And that it is necessary to maintain a long-term minimal concentrations of deslorelin (above a certain threshold) to maintain the chemical castration effect. Contrary to the prior art and the common belief concerning the mode of action of deslorelin, it is moreover clearly demonstrated a PK/PD relationship between the in vivo levels of deslorelin and the PD effects, allowing us to identify an efficacy threshold.

Accordingly, it is now demonstrated that in a non-human animal already chemically castrated, the ‘burst’ (also named herein after or “flare up”) effect is not necessary after all but it is necessary to maintain long-term concentrations of deslorelin above a certain threshold. In addition to this, it is shown that maintaining a range of concentration of deslorelin in the blood stream is necessary to obtain and keep the effects of castration. Contrary to the prior art and the common believes concerning the mode of action of deslorelin, it is moreover clearly demonstrated a PK/PD relationship between the in vivo levels of deslorelin and the PD effects, allowing us to identify an efficacy threshold.

### SUMMARY

The present invention regards to deslorelin, in a medicinal or a non-medicinal product, for its use in chemical castration of a non-human mammal within 1 month after administration, wherein upon administration, the Cmax of deslorelin (preferably at around 1 to 2 hours after administration) exceeds 7000 pg/mL and a deslorelin concentration in the non-human mammal plasma is at least 10 pg/mL. In a preferred embodiment, deslorelin is administered subcutaneously or intramuscularly to a non-human mammal in need thereof, at a dosage efficient to induce a deslorelin plasma concentration between 4000 pg/mL and 40 000 pg/mL between 1 h and 24 hours and a plasma concentration below 400 pg/ml from 8 days after administration.

In a specific embodiment of use, the deslorelin plasma concentration is between 10 pg/mL and 400 pg/ml from 8 days after administration up to at least 6 months.

In another embodiment, deslorelin is used for inducing temporary infertility in intact male animal over a period of several months, preferably at least 6 months, comprising administering deslorelin subcutaneously or intramuscularly to a mammal in need thereof, at a dosage efficient to induce a deslorelin plasma concentration between 4000 pg/mL and 40 000 pg/mL between 1 h and 24 hours and a plasma concentration below 400 pg/ml since 8 days after administration. In a preferred embodiment, the deslorelin plasma concentration is below 400 pg/ml from 8 days after administration up to 6 months. Preferably, after administration, deslorelin is released substantially continuously from 8 days after administration to at least 6 months at a level plasma concentration below 400 pg/mL.

In another embodiment, the invention concerns a sustained-release drug delivery system (DDS) containing deslorelin for its use as described above, administered to a non-human mammal by a subcutaneous or intramuscular route, wherein the sustained-release DDS releases for 2 days more than 15%, preferably more than 20%, preferably more than 25%, preferably more than 30%, preferably more than 35%, preferably more than 40%, preferably more than 45% and even preferably more than 50% of the deslorelin contained in the DDS and the remaining effective dose of deslorelin is released between 3 to 6 months or more. In a preferred embodiment, the DDS is a subcutaneous or intramuscular implant comprising deslorelin and a biodegradable polymer, for use in chemical castration of a non-human mammal, wherein the implant releases deslorelin with a Cmax of deslorelin after administration (preferably at around 1 or 2 hours after administration) that exceeds 7000 pg/mL and a deslorelin concentration in the non-human mammal plasma is at least 10 pg/mL and maintained for at least 6 months. In a preferred embodiment, deslorelin is administered to a non-human mammal in need thereof, at a dosage efficient to induce a deslorelin plasma concentration between 4000 pg/mL and 40 000 pg/mL between 1 h and 24 hours after administration and a plasma concentration below 400 pg/ml since 8 days after administration, preferably from 8 days after administration up to at least 6 months.

In another embodiment, the invention regards to a subcutaneous or intramuscular implant comprising deslorelin and a biodegradable polymer for inducing temporary infertility in intact male animal over a period of at least 6 months, comprising administering deslorelin subcutaneously or intramuscularly to a mammal in need thereof, at a dosage efficient to induce a deslorelin plasma concentration between 4000 pg/mL and 40 000 pg/mL between 1 h and 24 hours and a plasma concentration below 400 pg/ml since 8 days after administration.

In another embodiment, the invention regards to deslorelin for use in chemical castration of a non-human mammal, wherein upon administration, the deslorelin decreases the Testosterone levels in plasma of said non-human mammal below 0.4 ng/ml and a deslorelin concentration in the non-human mammal plasma is at least 10 pg/mL, preferentially above 15 pg/mL.

According to another embodiment, the invention provides a method for chemically castrating a non-human animal comprising the steps of:
(a) administering an effective amount of deslorelin so as to obtain a Cmax concentration between 7000 pg/mL and 40 000 pg/ml of plasma of deslorelin or an equivalent amount of a pharmaceutically acceptable salt thereof within first 24 h after injection; and thereafter,
(b) administering a maintenance dose so that mean concentration is above 10 pg/ml of plasma of deslorelin or an equivalent amount of a pharmaceutically acceptable salt thereof.

Preferentially, in step (a) and/or in step (b) a subcutaneous or intramuscular sustained-release Drug Delivery System (DDS) is injected. In a specific embodiment in step (b) a subcutaneous or intramuscular sustained-release Drug Delivery System (DDS) is injected at the same time of the implant of step (a). In a preferred embodiment, the sustained-release Drug Delivery System (DDS) of step (a) is a subcutaneous or intramuscular implant. Preferably, the implant is biodegradable polymer.

Another embodiment of invention regards to a method for the induction of temporary infertility in healthy, intact, sexually mature male animals, preferably dogs comprising the steps of:
(a) administering an effective amount of Deslorelin so as to obtain a Cmax concentration between 7000 pg/mL and 40 000 pg/ml of plasma of Deslorelin or an equivalent amount of a pharmaceutically acceptable salt thereof within first 24 h after injection; and thereafter
(b) administering a maintenance dose so that means concentration is above 10 pg/ml of plasma of Deslorelin or an equivalent amount of a pharmaceutically acceptable salt thereof.

Preferentially, in step (a) and/or in step (b) a subcutaneous or intramuscular sustained-release Drug Delivery System (DDS) is injected. In a specific embodiment in step (b) a subcutaneous or intramuscular sustained-release Drug Delivery System (DDS) is injected at the same time of the implant of step (a). In a preferred embodiment, the sustained-release Drug Delivery System (DDS) of step (a) is a subcutaneous or intramuscular implant. Preferably, the implant is biodegradable polymer.

Another embodiment of invention regards a method for the induction of temporary infertility to delay the first oestrus and heat signs, and to prevent pregnancy at a young age in intact and healthy sexually immature female animals (preferably in dogs) comprising the steps of:
(a) administering an effective amount of deslorelin so as to obtain a Cmax concentration between 7000 pg/mL and 40 000 pg/ml of plasma of deslorelin or an equivalent amount of a pharmaceutically acceptable salt thereof within first 24 h after injection; and thereafter
(b) administering a maintenance dose so that means concentration is above 10 pg/ml of plasma of deslorelin or an equivalent amount of a pharmaceutically acceptable salt thereof.

Preferentially, in step (a) and/or in step (b) a subcutaneous or intramuscular sustained-release Drug Delivery System (DDS) is injected. In a specific embodiment in step (b) a subcutaneous or intramuscular sustained-release Drug Delivery System (DDS) is injected at the same time of the implant of step (a). In a preferred embodiment, the sustained-release Drug Delivery System (DDS) of step (a) is a subcutaneous or intramuscular implant. Preferably, the implant is biodegradable polymer.

Another embodiment of invention regards a method for the induction of temporary infertility and suppression of urine odour and of sexual behaviours such as libido, vocalization, urine marking, and aggressiveness in intact male animals (preferably in cats), comprising the steps of:
(a) administering an effective amount of deslorelin so as to obtain a Cmax concentration between 7000 pg/mL and 40 000 pg/ml of plasma of deslorelin or an equivalent amount of a pharmaceutically acceptable salt thereof within first 24 h after injection; and thereafter
(b) administering a maintenance dose so that means concentration is above 10 pg/ml of plasma of deslorelin or an equivalent amount of a pharmaceutically acceptable salt thereof.

Preferentially, in step (a) and/or in step (b) a subcutaneous or intramuscular sustained-release Drug Delivery System (DDS) is injected. In a specific embodiment in step (b) a subcutaneous or intramuscular sustained-release Drug Delivery System (DDS) is injected at the same time of the implant of step (a). In a preferred embodiment, the sustained-release Drug Delivery System (DDS) of step (a) is a subcutaneous or intramuscular implant. Preferably, the implant is biodegradable polymer.

Still according to another embodiment, the present invention provides a method for diagnosing behavioral disorders (aggressiveness) in a non-human mammal treated with a deslorelin sustained release implant comprising the steps of:
a) taking a blood sample of said non-human mammal treated with a deslorelin,
b) measuring the testosterone plasma level in said blood sample,
c) determining the level of testosterone in said blood plasma, wherein said testosterone plasma level in said blood sample is above 0.2 ng/ml, preferentially 0.4 ng/ml,
d) finally, determining the dosage of deslorelin to be further administered to said non-human mammal for solving behavioral disorders.

In another embodiment, the invention regards to the use of deslorelin for inducing temporary infertility in intact male animal over a period of at least 6 months, comprising administering deslorelin subcutaneously or intramuscularly to a mammal in need thereof, at a dosage efficient to induce a deslorelin plasma concentration, wherein upon administration, the Cmax of deslorelin at 1 hour after administration exceeds 7000 pg/mL and a deslorelin concentration in the non-human mammal plasma is at least 10 pg/mL.

Another embodiment of invention regards to a contraceptive method of a non-human mammal comprising administering subcutaneously or intramuscularly to said non-human mammal, an implant comprising deslorelin and a biodegradable polymer, wherein the implant releases deslorelin with a Cmax of deslorelin at 1 hour after administration exceeding 7000 pg/mL and wherein a deslorelin concentration in the non-human mammal plasma is at least 10 pg/mL and maintained for at least 6 months. According to this embodiment, deslorelin is administered subcutaneously or intramuscularly to a non-human animal in need thereof, at a dosage efficient to induce a deslorelin plasma concentration between 4000 pg/mL and 40 000 pg/mL between 1 h and 24 hours and a plasma concentration below 400 pg/ml since 8 days after. Preferentially the deslorelin plasma concentration is below 400 pg/mL from 8 days after administration up to at least 6 months.

According to another embodiment, the invention provides a maintenance regimen for inhibiting or decreasing animal fertility comprising:
(a) first administering to an animal in need thereof, a therapeutically effective amount of a deslorelin product to induce a deslorelin plasma concentration between 4000 pg/mL and 40 000 pg/mL for a first predetermined period of time;
(b) subsequently administering to an animal in need of the therapeutically effective amount of a continuously releasing system dose of deslorelin for a second predetermined period of time to obtain a deslorelin plasma concentration of at least 10 pg/ml.

In another embodiment, the invention regards to deslorelin, for its use in prevention of behavioral disorder (including aggressiveness) in a non-human mammal, wherein deslorelin is administered subcutaneously or intramuscularly with a sustained release implant and wherein deslorelin concentration in the non-human mammal plasma is at least 10 pg/mL (for at least 6 months).

Another embodiment of the invention regards to deslorelin, for its use in prevention of FSH/LH levels impairments in a non-human mammal, wherein deslorelin is administered subcutaneously or intramuscularly with a sustained release implant and wherein deslorelin concentration in the non-human mammal plasma is at least 10 pg/mL (for at least 6 months).

Finally, the invention also generally relates to the use of deslorelin or a sustained release drug delivery system comprising deslorelin for the manufacturing of a medicament for:
inducing chemical castration of a non-human mammal, or
inducing temporary infertility in intact male animal over a period of several months, preferably in dogs, or
inducing of temporary infertility to delay the first oestrus and heat signs, and to prevent pregnancy at a young age in intact and healthy sexually immature female animals (preferably in dogs), or
inducing temporary infertility and suppression of urine odour and of sexual behaviours such as libido, vocalization, urine marking, and aggressiveness in intact male animals (preferably in cats), or
contraception or inhibiting or decreasing fertility in a non-human mammal, or
preventing behavioral disorder (including aggressiveness) in a non-human mammal, or
preventing FSH/LH levels impairments in a non-human mammal.

According to the invention, all embodiments described herein in this specification for the different uses of deslorelin and different methods and compositions involving the use of deslorelin also apply to the use of deslorelin for the manufacturing of the above-mentioned medicaments.

### BRIEF DESCRIPTION OF DRAWINGS

In addition to the exemplary aspects and embodiments described above, further aspects and embodiments of the invention will become apparent by reference to the drawings and by study of the following descriptions.

FIG. 1 depicts Testosterone Ratio Post/Pre GnRH stimulation. The ratio is measured at baseline and at Day 14 for the Control group (saline solution) and for the Treated Group (Suprelorin® 4.7) in cat males. This figure is corresponding to the results obtained according to Example 2.

FIG. 2 depicts the deslorelin influence on Sperm concentration in male cats. The Sperm concentration (/μL) measured over time in treated cats with Suprelorin® according to example 3.

FIG. 3 depicts the direct correlation between injected deslorelin concentration and testosterone concentration. The apparent relationship between plasma deslorelin concentrations and PD parameters over time was assessed graphically from the testosterone and deslorelin concentrations obtained in 16 healthy male cats treated with Suprelorin® 4.7 mg according to example 4.

FIG. 4 depicts the results of mean testicular volume over time curve in cat males treated with Suprelorin® 4.7 mg (n=5 control cats and n=10 treated cats) according to example 7. On this graph, the mean diameter of the mean testicular volume (right testicle) (in mm³) and its 95% confidence interval (CI) is expressed as a function of time (in weeks).

FIG. 5 depicts the mean ratios (and their 95% CI) of post/pre GnRH stimulation testosterone in treated dogs (n=6), measured over approximately 15 months of study according to example 9. On this figure, the mean ratios in expressed as a function of time (in days).

FIG. 6 depicts the results of mean ratios (and their 95% CI) post/pre-GnRH stimulation LH ratios in treated dogs (n=6) measured for approximately 15 study months according to example 10. On this figure, the mean ratios are expressed as a function of time (in days).

FIG. 7 depicts mean semi-logarithmic deslorelin plasma concentrations (pg/mL) and linear pre-GnRH hormone (testosterone, LH and FSH) serum concentrations (pg/mL) over the entire study (n=6 Treated dogs) as a function of time (days) according to example 11.

FIGS. 8A, 8B and 8C depict respectively the mean of percentages of efficacy results (FIG. 8A), the detailed levels of Testosterone concentration (in pg/mL) for study 1 (FIG. 8B) and for study 2 (FIG. 8C) according to example 12.

FIGS. 9A and 9B present respectively the mean of sum score of sexual behaviors for study 1 (FIG. 9A) and for study 2 (FIG. 9B) for study 2 example 12.

FIGS. 10A and 10B show respectively the mean vocalization variation for study 1 (FIG. 10A) and for study 2 (FIG. 10B) example 12.

FIGS. 11A and 11B show respectively the Urine marking, the % change from baseline is respectively for study 1 (FIG. 11A) and for study 2 (FIG. 11B) example 12.

FIGS. 12A and 12B show the urine odor variation/reduction measured as the change from baseline respectively for study 1 (FIG. 12A) and for study 2 (FIG. 12B) example 12.

FIGS. 13A and 13B show the % change of testicular volume respectively for study 1 (FIG. 13A) and for study 2 (FIG. 13B) example 12.

FIGS. 14A and 14B show the % change of appearance penile spines respectively for study 1 in (FIG. 14A) and for study 2 (FIG. 14B) example 12.

FIG. 15 illustrates the PK/PD preclinical study results in intact male cat (kitten) described in example 13. PK_study results are shown in FIG. 15; levels records of testosterone are presented in FIG. 15B; penile spines and behaviors records are presented in FIGS. 15 and 15D.

### DETAILED DESCRIPTION

Deslorelin is used as deslorelin acetate in a commercial implant named Suprelorin® 4.7 mg or 9.4 mg implant for dogs. It is prescribed for the induction of temporary infertility in healthy, entire, sexually mature male dogs. Deslorelin is a GnRH agonist, and acts by suppressing the function of the pituitary-gonadal axis when applied in a low, continuous dose. This suppression results in the failure of treated animals to synthesis and/or release Follicle Stimulating Hormone (FSH) and Luteinizing Hormone (LH), the hormones responsible for the maintenance of fertility.

The continuous low dose of deslorelin will reduce the functionality of the male reproductive organs, libido and spermatogenesis and lower the plasma testosterone levels, from 4 to 6 weeks after implantation. A short transient increase in plasma testosterone may be seen immediately after implantation. Measurement of plasma concentrations of testosterone has demonstrated the persistent pharmacological effect of the continuing presence of deslorelin in the circulation for at least 12 months following administration of the veterinary medicinal product.

As used here, a «behavioral disorder in a non-human mammal animal» includes but is not limited to urine odor, libido, vocalization, urine marking, as well as aggressiveness in intact male animals (preferably in cats).

As used herein, the term “chemical castration” refers to the use of chemicals or drugs to stop sex hormone production whether to reduce or suppress procreation and sexual activity. Chemical castration is sometimes called medical castration or hormone therapy. Chemical castration is reversible, that is to say that the hormone production resumes when the use of chemicals or drugs is stopped. Unlike surgical castration, where the gonads are removed through an incision in the body, chemical castration does not remove organs, nor is it a form of sterilization.

As used herein, the term «sexual activity» refers in particular to and encompasses sexual behaviours such as libido, vocalization, urine or scent marking and aggressiveness, whether in intact male or female animals. Courtship, mating and scent marking are also included as examples of sexual activity of intact male or female animals.

As used herein, the term «Cmax» of deslorelin means the maximum plasma concentration of deslorelin after its administration.

As used herein, the term “healthy animal” means an animal that has no contraindications for temporary infertility induction, surgical castration or chemical castration.

As used herein, the term “intact animal” refers to an animal which has not been surgically castrated and which is not currently under treatment for temporary infertility.

As used herein, the term “non-human animal” may refer to any organism of the kingdom Animalia except humans (Homo sapiens). Examples of “animals” as that term is used herein include, but are not limited to, companion animals, such as dogs, cats, and horses; and livestock animals, such as cows, goats, sheep, and pigs.

As used here, the term “pharmaceutical salt thereof” with reference to deslorelin means a pharmaceutically acceptable salt of deslorelin. An example of a pharmaceutical salt thereof is deslorelin acetate.

As used herein, the term “sexually mature female animal” means a female animal having seasonally estrus and heat signs.

On the contrary, a “sexually immature female animal” is a female animal which is not yet able to produce estrus and does not have any heat signs.

As used here, the term «temporary» when referred to infertility means that the infertility is for a certain period of time, i.e. the infertility is reversible.

As used herein, the term “temporary infertility”, with reference to a chemical that can induce it, refer to an inability to conceive. Some chemotherapy treatment can cause infertility while one is having the treatment. But fertility can come back some times after treatment has finished. This will depend on the drugs, but also on other factors such as age and whether the patient is male or female. According to the meaning of the invention, “temporary infertility” may thus be assimilated to contraception or to the result of a contraceptive method.

The term «Tmax» as used herein means the time at which the maximal plasma concentration (Cmax) of deslorelin is achieved or observed after administration of deslorelin.

### Pharmaceutical Compositions

One aspect of the disclosure provides a composition for use in inducing chemical castration in a non-human animal in need thereof, comprising a therapeutically effective amount of deslorelin and an excipient, as set out in the claims.

In a preferred embodiment of the present invention, the deslorelin is formulated in a solid implant formulation.

In a preferred embodiment, said implant formulation comprises stearin (also known as hydrogenated palm oil) and lecithin.

According to this particular embodiment of the present invention, the solid implant formulation preferably comprises about 2-12% (w/w) deslorelin (on an active basis), about 0.5-2.5% (w/w) lecithin and about 85-97.5% (w/w) stearin.

More preferably, the solid implant formulation comprises about 4-10% (w/w) deslorelin (on an active basis), about 0.5-1.5% (w/w) lecithin and about 85-94% (w/w) stearin.

Particularly preferred solid implant formulations are selected in the group comprising formulations comprising 85-89% (w/w) stearin, 9-10% (w/w) deslorelin (on an active basis) and 0.8-1.2% (w/w) lecithin.

In a further preferred embodiment, said solid implant formulation may further comprise sodium acetate anhydrous.

Stearin is partially hydrogenated palm oil. Its principle fatty acids are C16:0 (45%) and C18:0 (53%). Melting point is about 55° C.

Lecithin is phosphatidylcholine. It is a mixture of diglycerides of stearic, palmitic and oleic acids linked to the choline ester of phosphoric acid.

In another preferred embodiment, the deslorelin is formulated in a liquid form composition.

Deslorelin may be administered by any suitable means, for example by implant preferentially biodegradable over time, by sustained-release drug delivery systems (DDS), by biocompatible polymer-solvent systems comprising a biodegradable polymer, compatible with a subcutaneous, dermal or intramuscular administration.

According to the invention, “DDS” refers to any system enabling the delivery of a drug in the organism. Preferred DDS according to the invention includes any type of implants; in particular, this term encompasses liquid and solid implants, such implants being with at least a polymeric or non-polymeric material.

According to the invention, “sustained-release” when referred to DDS means that the deslorelin is not released as a single administration of the total dose of deslorelin. On the contrary, the sustained-release DDS enables to deliver deslorelin over a certain amount of time as disclosed in the present application.

In some embodiments, the implant is preferentially biodegradable over time. It is selected from the group consisting of biodegradable materials and bioerodible dosage forms.

As used herein, the term “biocompatible” may mean “not harmful to living tissue.”

This term encompasses both biodegradable and bioerodible.

As used herein, “degradable” means that the polymer will degrade or erode in vivo to form smaller chemical species, wherein the degradation can result, for example, from enzymatic, chemical (“biodegradable” polymer), and/or physical processes («bioerodible” polymer).

As used herein, the term “biodegradable” may refer to any water-insoluble material that may be converted under physiological conditions into one or more water-soluble materials, without regard to any specific degradation mechanism or process.

In some embodiments, the biodegradable material is a biodegradable polymer (or biopolymer), preferably selected from the group consisting of polylactic acid, polyglycolic acid, polylactide, polyglycolide, polycaprolactones, polyanhydrides, polyamides, polyurethanes, polyesteramides, poly orthoesters, polydioxanones, polyacetals, polyketals, polycarbonates, polyphosphazenes, polyhydroxybutyrates, polyhydroxyvalerates, polyalkylene oxalates, polyalkylene succinates, poly(malic acid), polyethylene glycol, hyaluronic acid, chitin and chitosan, a copolymer thereof, a terpolymer thereof, and any combination thereof.

In some embodiments, the biodegradable polymer comprises monomers that are selected from the group consisting of lactide, glycolide, caprolactone, p-dioxanone, trimethylene carbonate, 1,5-dioxepan-2-one, 1,4-dioxepan-2-one, ethylene oxide, propylene oxide, sebacic anhydride, diketene acetals/diols, lactic acid, and combinations thereof.

As used herein, the term “implant” may also be understood as a bioerodible dosage form and includes devices such as microspheres; “liquid polymer depot-type” formulations (or in-situ forming implants); and solid polymeric implants, such as pellets or mini-pellets. The implant comprises deslorelin and a bioerodible polymer as excipient.

As used herein, the term “bioerodible” may refer to any water-insoluble material that may be eroded mechanically via biological processes that solubilize at least partially the material.

Upon erosion, the bioerodible polymer will release deslorelin in the body fluids, including the blood and lymph or tissues. Illustrating examples of bioerodible polymers according to the invention include polyhydroxy acids, such as poly(lactide)s, poly(glycolide)s, poly(lactide-co-glycolide)s, poly(lactic acid)s, poly(glycolic acid)s, and poly(lactic acid-co-glycolic acid)s, polyanhydrides, polyorthoesters, polyetheresters, polyethylene glycol, poly-e-caprolactone, polyesteramides, polyphosphazines, polycarbonates, polyamides, and copolymers and blends thereof, fats, waxes and triglyceryl esters of long chain C₁₂-C₂₂ fatty acids, such as stearates, palmitates, laurates, myristates, arachidates and behenates, and mixtures thereof, having melting points greater than 50° C., preferably glyceryl tristearate. Preferably, the polymer is selected from the group consisting in poly-e-caprolactone (PCL), poly(lactide)s, poly(glycolide)s, and copolymers or blends thereof such as poly(lactide-co-glycolide) s and glyceryl tristearate.

The injectable implant may further comprise an antioxidant. Antioxidants useful for injectable implants are known in the art. One preferred anti-oxidant is BHT (butylated hydroxytoluene). Such implants, microspheres and in-situ forming implants can be manufactured using methods well known in the art, for example described in patent applications EP3349726, WO90/03768, WO2006/063794, WO2009/091737, WO98/07423, EP1197207, EP0525307, WO98/08533, WO2020/222399, WO2020/130585 and WO2013/082373, without dissolution of deslorelin during the process. In a preferred embodiment, the present invention concerns an implant as defined above, comprising deslorelin, a bioerodible polymer selected from the group consisting in poly-e-caprolactone (PCL), poly(lactide)s, poly(glycolide)s, poly(lactide-co-glycolide)s, copolymers or blends thereof, and glyceryl tristearate, and optionally an antioxidant.

In some embodiments, the deslorelin is present in the liquid form composition in a dosage effective for greater than one month, more preferred greater than two months and most preferred greater than three months or greater. In a preferred embodiment, the deslorelin is present in the composition in a dosage effective for greater than four months, more preferred greater than five months and most preferred greater than six months or greater.

As used herein, the term “liquid” may refer to the ability of a composition to undergo deformation under a shearing stress, regardless of the presence or absence of a non-aqueous solvent.

Liquid compositions according to the present disclosure have a liquid physical state at ambient and body temperatures and remain liquid in vivo, i.e., in a largely aqueous environment.

The deslorelin composition is formulated to be released continuously, preferentially in a uniform manner by the slow delivery system. To this aim, the viscosity of the deslorelin liquid composition at ambient temperature (i.e. about 20° C.) is greater than about 0.5 Pa s, greater than about 1.0 Pa s, greater than about 2.0 Pa s, greater than about 3.0 Pa s, greater than about 4.0 Pa s, greater than about 5.0 Pa s, greater than about 6.0 Pa s, greater than about 7.0 Pa s, greater than about 8.0 Pa s, greater than about 9.0 Pa s, greater than about 10.0 Pa s, greater than about 11.0 Pa s, greater than about 12.0 Pa s, greater than about 13.0 Pa s, greater than about 14.0 Pa s, greater than about 15.0 Pa s, greater than about 16.0 Pa s, greater than about 17.0 Pa s, greater than about 18.0 Pa s, greater than about 19.0 Pa s, or greater than about 20.0 Pa s. In some further embodiments, the viscosity of the substantially homogeneous composition may be greater than about 30.0 Pa s, greater than about 40.0 Pa s, greater than about 50.0 Pa s, greater than about 60.0 Pa s, greater than about 70.0 Pa s, greater than about 80.0 Pa s, greater than about 90.0 Pa s, greater than about 100.0 Pa s, greater than about 110.0 Pa s, greater than about 120.0 Pa s, greater than about 130.0 Pa s, greater than about 140.0 Pa s, greater than about 150.0 Pa s, greater than about 160.0 Pa s, greater than about 170.0 Pa s, greater than about 180.0 Pa s, greater than about 190.0 Pa s, or even greater than about 200.0 Pa s.

Alternatively, the viscosity of the substantially homogeneous composition may be greater than any whole number from about 0.50 to about 200.0 Pa s.

Viscosity can be measured using a suitable viscometer in a setup that is compatible for the deslorelin liquid composition. For example, viscosity can be measured using a viscometer such as, but not limited to, a Brookfield Viscometer or an Anton Paar Rheoplus viscometer with an appropriate setup. Whenever a viscosity value is mentioned/defined in this document, it concerns the viscosity as measured using a Brookfield. Viscometer à 20° C.

A variety of excipients commonly used in formulation and preferentially in pharmaceutical formulations may be selected on the basis of several criteria such as, for example, the desired dosage form and the release profile properties of the dosage form. Non-limiting examples of suitable excipients include an agent selected from the group comprising a binder, a filler, a preservative, a diluent, a lubricant, dispersing agent, a pH modifier, a stabilizer, and combinations of any of these agents.

### Methods for Using a Deslorelin Composition

Another aspect of the present disclosure encompasses a method for inducing chemical castration in a non-human animal in need thereof. The method comprises administering to the non-human animal a composition comprising an effective amount of deslorelin.

In one embodiment, the effective amount of deslorelin is the dosage of Suprelorin® implant of 4.7 mg and 9.4 mg.

A first object of the present invention is deslorelin, for its use in chemical castration of a non-human mammal within 1 month after administration, wherein upon administration, the Cmax of deslorelin after administration exceeds 7000 pg/mL and a deslorelin concentration in the non-human mammal plasma is at least 10 pg/mL.

The administration of deslorelin to an animal achieves a Cmax of deslorelin of about 7000 pg/mL to about 200 000 pg/mL at a time (Tmax) of about 0.4 to about 3.4 hours depending of animal species, such as a Cmax of deslorelin of about 7500 pg/mL to about 170 000 pg/mL. In a specific example, the Cmax of deslorelin at 1 hour after administration exceeds 7429 pg/mL.

In other embodiments, the Cmax of deslorelin may also be achieved at a Tmax of about 0.7 to about 2 hours, such as a Tmax of about 0.5 to about 1.7 hours. In some embodiments, the Cmax may vary in different non-human animals, therefore the Cmax could be even higher. For example, the Cmax may be about 7200 pg/mL, about 7400 pg/mL, about 8000 pg/mL, about 9000 pg/mL, about 10000 pg/mL, about 11000 pg/mL, about 12000 pg/mL, about 13000 pg/mL, about 15000 pg/mL, about 20000 pg/mL, about 25000 pg/mL, about 30000 png/mL, about 35000 pg/mL, about 40000 pg/mL, about 45000 pg/mL, about 50000 pg/mL, about 55000 pg/mL, about 60000 pg/mL, about 65000 pg/mL, about 70000 pg/mL, about 75000 pg/mL, about 80000 pg/mL, about 85000 pg/mL, about 90000 pg/mL, about 95000 pg/mL, or about 100 000 pg/mL, or about 110 000 pg/mL, or about 120 000 pg/mL, or about 130 000 pg/mL, or about 140 000 pg/mL or about 150 000 pg/mL or about 160 000 pg/mL, or about 170 000 pg/mL. In exemplary embodiments, the Cmax of deslorelin may be 375 ng/ml to 10000 ng/ml. In some embodiments, the Cmax of deslorelin may be greater than 500 pg/mL. In other embodiments, the Cmax of deslorelin may be less than 300000 pg/mL.

In other embodiments, the Tmax may occur in an individual non-human animal at a 30-minute, 1-hour, or 2-hour time interval, with the range being from about 30 minutes to 2 hours to reach Tmax. The Tmax may be about 0.4 hour, about 0.5 hour, about 0.6 hour, about 0.7 hour, about 0.8 hour, about 0.9 hour, about 1.0 hour, about 1.1 hours, about 1.2 hours, about 1.3 hours, about 1.4 hours, about 1.5 hours, about 1.6 hours, about 1.7 hours, about 1.8 hours, about 1.9 hours, about 2.0 hours, about 2.2 hours, about 2.4 hours, about 2.6 hours, about 2.8 hours, about 3.0 hours, about 3.2 hours, or about 3.4 hours. In one embodiment, the Tmax may be from 0.4 to 4 hours. In other exemplary embodiments, the Tmax may be from 0.5 to 1.0 hour. In some embodiments, the Tmax may be greater than 0.4 hour. In other embodiments, the Tmax may be less than 3.4 hours. In exemplary embodiments, the Tmax may be from 0.03 day to 1.33 days.

The area under the curve (AUC) may range from about 10 000 day*pg/mL to about 110 000 day*pg/mL. In exemplary embodiments, the AUC may range from about 11000 day*pg/mL to about 15000 day*pg/mL, from about 15000 day*pg/mL to about 20000 day*pg/mL, from about 20000 day*pg/mL to about 25000 day*pg/mL, from about 25000 day*pg/mL to about 30000 day*pg/mL, from about 30000 day*pg/mL to about 35000 day*pg/mL, from about 35000 day*pg/mL to about 40000 day*pg/mL, from about 40000 day*pg/mL to about 45000 day*pg/mL, from about 45000 day*pg/mL to about 50000 day*pg/mL, from about 50000 day*pg/mL to about 55000 day*pg/mL, from about 55000 day*pg/mL to about 60000 day*pg/mL.

According to the first object of the present invention, the deslorelin is administered subcutaneously or intramuscularly to a non-human mammal in need thereof, at a dosage efficient to induce a deslorelin plasma concentration between 4000 pg/ml and 40 000 pg/mL between 1 h and 24 hours, preferably between 7000 pg/mL and 40 000 pg/mL between 1 h and 24 hours, and a plasma concentration below 400 pg/ml from 8 days after administration. Preferably, the deslorelin plasma concentration is between 10 pg/mL and 400 pg/ml from 8 days after administration up to at least 6 months.

According to a particular embodiment of the first object of the present invention, deslorelin is used for inducing temporary infertility.

Therefore, the present invention also relates to deslorelin, for its use in inducing temporary infertility in intact male animal over a period pf at least 6 month, comprising administering deslorelin subcutaneously or intramuscularly to a mammal in need thereof, at a dosage efficient to induce a deslorelin plasma concentration between 4000 pg/mL and 40 000 pg/mL between 1 h and 24 hours, preferably between 7000 pg/mL and 40 000 pg/mL between 1 h and 24 hours, and a plasma concentration below 400 pg/ml since 8 days after administration. Preferably, the deslorelin plasma concentration is below 400 pg/ml from 8 days after administration up to 6 months. According to this particular embodiment, after administration, the deslorelin is released substantially continuously from 8 days after administration to at least 6 months at a level plasma concentration below 400 pg/mL.

In addition, the invention also relates to the deslorelin, for its use in inducing temporary infertility to delay the first oestrus and heat signs, and to prevent pregnancy at a young age in intact and healthy sexually immature female animals comprising the steps of:
(a) administering an effective amount of deslorelin so as to obtain a Cmax concentration of deslorelin or an equivalent amount of a pharmaceutically acceptable salt thereof between, between 4000 pg/mL and 40000 pg/ml of plasma within first 24 h after injection, preferably between 7000 pg/mL and 40000 pg/ml of plasma within first 24 h after injection; and thereafter
(b) administering a maintenance dose of deslorelin so that means concentration of deslorelin or an equivalent amount of a pharmaceutically acceptable salt thereof is above 10 pg/ml of plasma.

Preferably, when deslorelin is used for inducing chemical castration or temporary infertility according to the first object of the present invention, deslorelin is formulated in the form of a sustained-release drug delivery system (DDS).

A second object of the present invention is therefore a sustained-release drug delivery system (DDS) containing deslorelin for its use in chemical castration of non-human mammal according to the first object of the present invention, wherein the DDS releases deslorelin with a Cmax of deslorelin after administration that exceeds 7000 pg/mL and a deslorelin concentration in the non-human mammal plasma is at least 10 pg/mL and maintained for at least 6 months. Preferably, the DDS is a subcutaneous or intramuscular implant comprising deslorelin and a biodegradable material such as a biodegradable polymer.

According to a particular embodiment of this second object, upon administration, the deslorelin decreases the Testosterone levels in plasma of said non-human mammal below 0.4 ng/ml and a deslorelin concentration in the non-human mammal plasma is at least 10 pg/mL, preferentially above 15 pg/mL.

A third object of the present invention is a sustained-release drug delivery system (DDS) containing deslorelin for its use in inducing temporary infertility in intact male animal over a period of at least 6 months, comprising administering deslorelin subcutaneously or intramuscularly to a mammal in need thereof, at a dosage efficient to induce a deslorelin plasma concentration between 4000 pg/mL and 40 000 pg/mL between 1 h and 24 hours, preferably between 7000 pg/mL and 40000 pg/ml of plasma within first 24 h after injection, and a plasma concentration below 400 pg/ml since 8 days after administration.

A fourth object of the present invention is also a method for chemically castrating a non-human mammal comprising the steps of:
(a) administering an effective amount of deslorelin so as to obtain a Cmax concentration of at least 7000 pg/mL of plasma, preferably between 7000 pg/mL and 40 000 pg/ml of plasma, of deslorelin or an equivalent amount of a pharmaceutically acceptable salt thereof within first 24 h after administration; and thereafter
(b) administering a maintenance dose so that means concentration of deslorelin or an equivalent amount of a pharmaceutically acceptable salt thereof is above 10 pg/ml of plasma.

According to a particular embodiment of this fourth object of the present invention, in step (a) and/or in step (b) a subcutaneous or intramuscular sustained-release Drug Delivery System (DDS) is injected. The sustained-release DDS of step (a) is preferably a subcutaneous or intramuscular implant. According to a preferred manner, in step (b) a subcutaneous or intramuscular sustained-release Drug Delivery System (DDS) is injected at the same time of the implant of step (a). Said implant preferably comprises a biodegradable material, such as a biodegradable polymer.

A fifth object of the present invention is a method for the induction of temporary infertility in healthy, intact, sexually mature male animals, preferably dogs, comprising the steps of:
(a) administering an effective amount of deslorelin so as to obtain a Cmax concentration of deslorelin or an equivalent amount of a pharmaceutically acceptable salt thereof, between 4000 pg/mL and 40000 pg/ml of plasma, preferably between 7000 pg/mL and 40 000 pg/ml of plasma within first 24 h after administration; and thereafter
(b) administering a maintenance dose of deslorelin so that means concentration is above 10 pg/ml of plasma of deslorelin or an equivalent amount of a pharmaceutically acceptable salt thereof.

According to a particular embodiment of this fifth object of the invention, in step (a) and/or in step (b) a subcutaneous or intramuscular sustained-release Drug Delivery System (DDS) is injected. In that case, the DDS preferably releases deslorelin with a Cmax of deslorelin at 1 hour after administration exceeding 7000 pg/mL and a deslorelin concentration in the non-human mammal plasma is at least pg/mL and maintained for at least 6 months. More preferably, deslorelin is administered at a dosage efficient to induce a deslorelin plasma concentration between 4000 pg/mL and 40 000 pg/mL between 1 h and 24 hours and a plasma concentration below 400 pg/ml since 8 days after, preferentially up to at least 6 months. According to a preferred manner, in step (b) a subcutaneous or intramuscular sustained-release Drug Delivery System (DDS) is injected at the same time of the implant of step (a). Said implant preferably comprises a biodegradable material, such as a biodegradable polymer.

The invention also contemplates the use of deslorelin for inducing temporary infertility in intact male animal over a period of at least 6 months, comprising administering deslorelin subcutaneously or intramuscularly to a mammal in need thereof, at a dosage efficient to induce a deslorelin plasma concentration, wherein upon administration, the Cmax of deslorelin at 1 hour after administration exceeds 7000 pg/mL and a deslorelin concentration in the non-human mammal plasma is at least 10 pg/mL.

A sixth object of the present invention is a method for the induction of temporary infertility to delay the first oestrus and heat signs, and to prevent pregnancy at a young age in intact and healthy sexually immature female animals (preferably in dogs) comprising the steps of:
(a) administering an effective amount of deslorelin so as to obtain a Cmax concentration of deslorelin or an equivalent amount of a pharmaceutically acceptable salt thereof between 4000 pg/mL and 40000 pg/ml of plasma within first 24 h after injection, preferably between 7000 pg/mL and 40000 pg/ml of plasma within first 24 h after injection; and thereafter
(b) administering a maintenance dose of deslorelin so that means concentration of deslorelin or an equivalent amount of a pharmaceutically acceptable salt thereof is above 10 pg/ml of plasma.

According to a particular embodiment of this sixth object of the invention, in step (a) and/or in step (b) a subcutaneous or intramuscular sustained-release Drug Delivery System (DDS) is injected. In that case, the DDS should be preferentially administered between 12 and 16 weeks of age. The DDS comprises deslorelin and a biodegradable material such as a biodegradable polymer, and the DDS preferably releases deslorelin with a Cmax of deslorelin at 1 hour after administration exceeding 7000 pg/mL and a deslorelin concentration in the non-human mammal plasma is at least 10 pg/mL and maintained for at least 6 months. Further, deslorelin is even more preferably administered at a dosage efficient to induce a deslorelin plasma concentration between 4000 pg/mL and 40 000 pg/mL between 1 h and 24 hours and a plasma concentration below 400 pg/ml since 8 days after, preferentially up to at least 6 months. According to a preferred manner, in step (b) a subcutaneous or intramuscular sustained-release Drug Delivery System (DDS) is injected at the same time of the implant of step (a). Said implant preferably comprises a biodegradable material, such as a biodegradable polymer.

A seventh object of the present invention is a method for the induction of temporary infertility and suppression of urine odour and of sexual behaviours such as libido, vocalization, urine marking, and aggressiveness in intact male animals, (preferably in cats), comprising the steps of:
(a) administering an effective amount of deslorelin so as to obtain a Cmax concentration between 4000 pg/mL and 40 000 pg/ml of plasma of deslorelin or an equivalent amount of a pharmaceutically acceptable salt thereof within first 24 h after injection, preferably between 7000 pg/mL and 40 000 pg/ml of plasma of deslorelin or an equivalent amount of a pharmaceutically acceptable salt thereof within first 24 h after injection; and thereafter
(b) administering a maintenance dose so that means concentration is above 10 pg/ml of plasma of deslorelin or an equivalent amount of a pharmaceutically acceptable salt thereof.

According to this seventh object of the present invention, said intact male animals are preferably cats, even more preferably cats from 3 months of age.

According to a particular embodiment of this seventh object of the invention, in step (a) and/or in step (b) a subcutaneous or intramuscular sustained-release Drug Delivery System (DDS) is injected. In that case, the DDS comprises preferably deslorelin and a biodegradable material, such as a biodegradable polymer, and the DDS releases deslorelin with a Cmax of deslorelin at 1 hour after administration exceeding 7000 pg/mL and a deslorelin concentration in the non-human mammal plasma is at least 10 pg/mL and maintained for at least 6 months. More preferably, deslorelin is administered at a dosage efficient to induce a deslorelin plasma concentration between 4000 pg/mL and 40 000 pg/mL between 1 h and 24 hours and a plasma concentration below 400 pg/ml since 8 days after, preferentially up to at least 6 months. According to a preferred manner, in step (b) a subcutaneous or intramuscular sustained-release Drug Delivery System (DDS) is injected at the same time of the implant of step (a). Said implant preferably comprises a biodegradable material, such as a biodegradable polymer.

A eighth object of the present invention is a method for diagnosing behavioral disorders (aggressiveness) in a non-human mammal treated with a deslorelin sustained release implant comprising the steps of:
a) measuring the Testosterone plasma level in a blood sample of said non-human mammal treated with said deslorelin sustained release implant,
b) determining the level of Testosterone in said blood plasma, wherein testosterone plasma level in said blood sample is above 0.2 ng/ml preferentially 0.4 ng/ml,
c) finally, determining the dosage of deslorelin to be further administered to said non-human mammal for solving behavioral disorders.

A ninth object of the present invention is a contraceptive method of a non-human mammal comprising administering subcutaneously or intramuscularly to said non-human mammal, an implant comprising deslorelin and a biodegradable material, such as a biodegradable polymer, wherein the implant releases deslorelin with a Cmax of deslorelin at 1 hour after administration exceeding 7000 pg/mL and wherein a deslorelin concentration in the non-human mammal plasma is at least 10 pg/mL and maintained for at least 6 months.

According to the this ninth object, the deslorelin is administered to a non-human mammal in need thereof, at a dosage efficient to induce a deslorelin plasma concentration between 4000 pg/mL and 40000 pg/mL between 1 h and 24 hours, preferably between 7000 pg/mL and 40000 pg/mL between 1 h and 24 hours, and a plasma concentration below 400 pg/ml since 8 days after administration. Preferably, the plasma concentration of deslorelin is below 400 pg/ml since 8 days after administration up to at least 6 months.

A tenth object of the invention is a maintenance regimen for inhibiting or decreasing animal fertility comprising:
(a) first administering to an animal in need thereof, a therapeutically effective amount of a deslorelin product to induce a plasma concentration of deslorelin or an equivalent amount of a pharmaceutically acceptable salt thereof between 4000 pg/mL and 40000 pg/mL for a first predetermined period of time, preferably between 7000 pg/mL and 40000 pg/mL;
(b) subsequently administering to an animal in need of the therapeutically effective amount of a continuously releasing system dose of deslorelin for a second predetermined period of time to obtain a plasma concentration deslorelin or an equivalent amount of a pharmaceutically acceptable salt thereof of at least 10 pg/ml.

An eleventh object of the present invention is deslorelin for its use in prevention of behavioral disorder in a non-human mammal, wherein deslorelin is administered subcutaneously or intramuscularly with a sustained release drug delivery system and wherein deslorelin concentration in the non-human mammal plasma is at least 10 pg/mL (for at least 6 months).

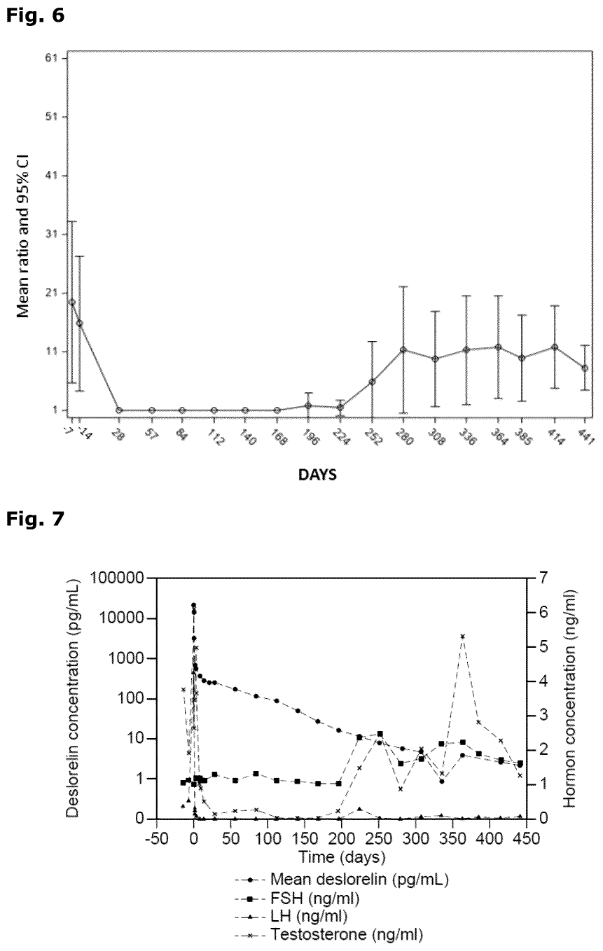

One of the embodiments regards the deslorelin administration to induce variations in hormonal pathways. Indeed, through the new data generated it is now apparent that deslorelin influences on LH concentration. Indeed, Example 10 shows the variation of LH hormone via results of Mean (95% CI) post/pre-GnRH stimulation LH ratios in treated dogs (n=6) measured for approximately 16 Study Months and presented in FIG. 6.

In addition, in example 11, it is shown the correlation between the concentration of injected deslorelin and the concentration of the hormones LH, FSH and testosterone in male cats treated with Suprelorin®. This study showed that there was an inverse correlation between deslorelin and testosterone/LH and FSH. Indeed, as soon as the deslorelin concentration became low (12 pg/mL), the other concentrations started to increase.

Therefore, a twelfth object of the present invention is deslorelin for its use in chemical castration of a non-human mammal, wherein upon administration, the deslorelin decreases the Testosterone levels in plasma of said non-human mammal below 0.4 ng/ml and a deslorelin concentration in the non-human mammal plasma is at least 10 pg/mL, preferentially above 15 pg/mL.

A thirteenth object of the present invention is deslorelin for its use in prevention of follicle stimulating hormone (FSH) and luteinizing hormone (LH) impairments in non-human animal, wherein deslorelin is administered subcutaneously or intramuscularly with a sustained release implant and wherein deslorelin concentration in the non-human mammal plasma is at least 10 pg/mL (for at least 6 months).

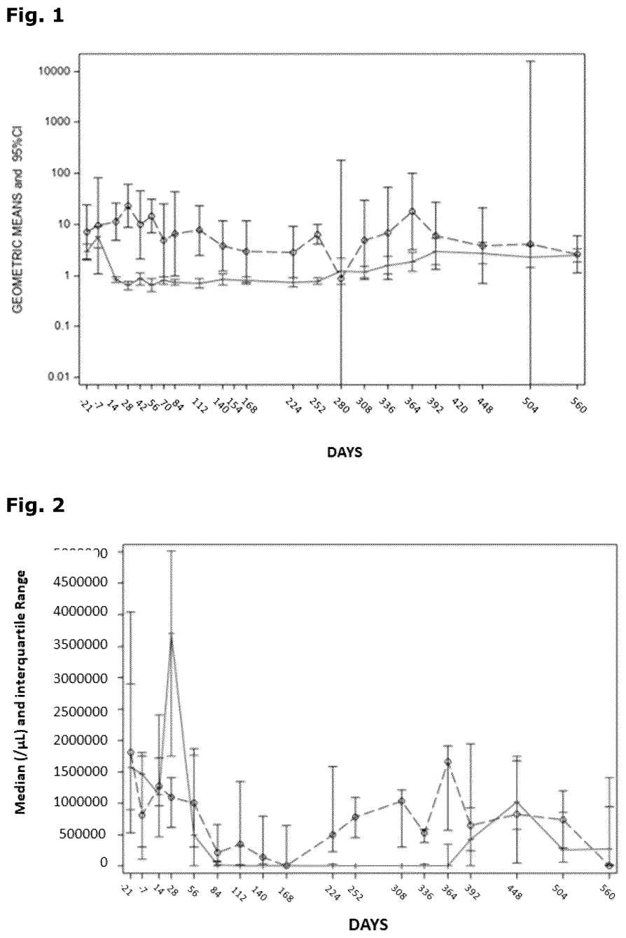

Indeed, it is now demonstrated that deslorelin impacts hormonal pathways. However, this correlation goes beyond the hormonal area. The hormonal pathway influences also the metabolic area in varying/amending or altering several metabolic pathways such as:
Sperm concentration: As shown in example 3 and FIG. 2, deslorelin has an impact on sperm concentration.
Testis weight: As shown in example 6 and FIG. 4, deslorelin has an impact on testis weight. Indeed, it has been shown that with deslorelin implant injection, the testis volume is reduced over time when deslorelin effect is present.
Fat and carbohydrate variation: The low level of Testosterone induces weight gain.
Urine odor improvement/reduction:

Urine marking is the normal and deliberate deposition of urine as a:
Pheromonal signal to other animals; especially unneutered male cats
Territorial signal
Sign of stress or arousal related to the social or physical environment.

In this context, urine smells pungent and it is desirable to removing or masking the smell. Several commercially available enzymatic urine odor neutralizers that should be used on surfaces after washing the area with mild detergent can be used for removing the odor but they are fastidious. Neutering or spaying is a proven treatment particularly for cats who mark as a reproductive advertisement.

One of the reason for surgical castration is to alleviate the strong and persistent urine odor in male animal, particularly with cat and ferret. It is known that after surgical castration, the urine odor is improved in few days.

One of the purpose of the present application is to show that deslorelin (also known as Suprelorin®) administration decreases urine and/or feces odor. Surprisingly, it has been shown by the inventors that odor reduction is mediated via at least two pathways. Indeed, one of such pathway is the testosterone-mediated sex hormone pathway. The inventors have also demonstrated another pathway unrelated to sex hormones. Therefore, the decrease in urine odor in an intact male animal receiving deslorelin according to the invention is improved when compared to the decrease in urine odor occurring in a surgically castrated male animal, especially in cats.

It is one aspect of the invention to alleviate urine odor in animal such as domestic animal in particular cat and preferably in unneutered male cats.

One of the purpose of the present application is to show that deslorelin/Suprelorin® administration decreases urine odor via the decrease of Testosterone/felinine and its metabolites levels.

It is known from prior art (Hendriks W H et al., Testosterone increases urinary free felinine, N-acetylfelinine and methylbutanolglutathione excretion in cats (Felis catus). J Anim Physiol Anim Nutr (Berl). 2008 February; 92 (1): 53-62) that testosterone increases the free excretion of feline, N-acetylfelinine and 3-methylbutanolglutathione in neutered adult male cats and intact females, whereas estradiol does not modulate this effect.

Cauxin, a carboxylesterase excreted as a major urinary component, regulates felinine production. It is also known that cauxin excretion is sex-dependent. In mature cats, cauxin excretion was higher in intact males than in castrated males or in intact or spayed females. Daily cauxin excretion decreased immediately after castration. Immunohistochemistry confirmed that cauxin expression in the kidney proximal straight tubules was higher in intact males than in castrated males. These results suggest that 1) cauxin excretion is regulated by sex hormones, such as testosterone, 2) cauxin functions as an esterase in the urine rather than in kidney cells, and 3) the decomposition products by cauxin are excreted in a species-, sex, and age-dependent manner, as is cauxin itself.

Upon castration, cauxin excretion decreased immediately, and the cause was confirmed to be a decrease in the level of cauxin expression in the proximal straight tubules. The plasma testosterone concentration in intact male cats has been reported to increase with age to 1.5, 6.8, and 12.6 pmol/mL at 6, 12, and 16 months after birth, respectively, and to decrease to zero after castration (Miyazaki et al, Chemical Signals in Vertebrates, 1998, 11, pp 51-60). It is thus postulated that the transcriptional activity of cauxin is regulated by sex hormones, such as testosterone.

Cauxin regulates felinine production. In vitro enzyme assays indicated that cauxin hydrolyzed the felinine precursor 3-methylbutanolcysteinylglycine to felinine and glycine.

Hence, the chemical castration induced by deslorelin release impacts cauxine and felinine production as well which is also responsible of urine odor. As felinine and N-acetylfelinine were detected in cat bile and fecal samples, indicating that felinine is excreted into the feces via bile from the liver (LC-MS/MS quantification of felinine metabolites in tissues, fluids, and excretions from the domestic cat (Felis catus), Futsuta et al, J Chromatogr B Analyt Technol Biomed Life Sci., 2018 Jan. 1; 1072:94-99).

In addition to the sex hormone pathway mediated by testosterone responsible for the odor of urine and/or faeces, the inventors have also discovered another pathway unrelated to sex-dependent hormones. Indeed, as shown in the examples, the inventors have identified new molecules as being molecules responsible of urine and or feces odor. These molecules are secreted outside the sexual hormone pathway. In fact, those molecules are not derivated from Testosterone cascade.

Therefore, one aspect of the present invention is the use of deslorelin to modify or alter or to down regulate or up regulate the levels of at least one molecule selected from the list of crotonic acid; indole; paracresol; cadaverine and derivatives thereof like acetylcadaverine, glutathionylaminopropylcadaverine; putrescine and derivatives like N-acetylputrescine, p-thereof coumaroylputrescine; mercapto derivatives like 2-mercapto-3-butanone, 2-mercaptoethanol, 3-mercaptohexyl butyrate, mercaptopyruvic acid, 3-mercaptopropionic acid, 3-mercapto-3-methylbutan-1-ol, 7-mercaptoheptanoylthreonine, 1alpha, 5alpha-dimercaptoandrostane-3alpha, 17beta-diol, mercaptopyruvic acid; taurine; isovalthine and isobuteine.

It is thus one objective of the invention to reduce urine and or feces odor by administering deslorelin to a non-human animal.

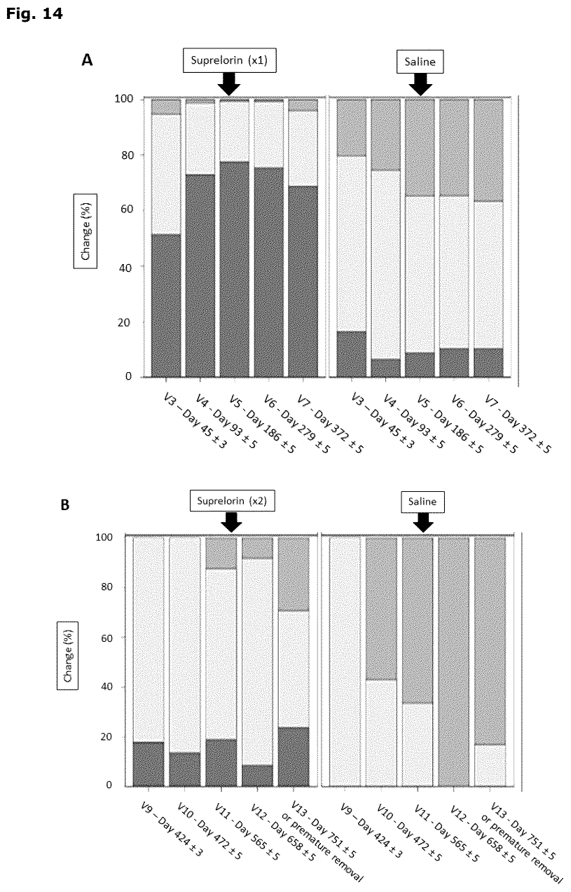

In another aspect of the invention, deslorelin administration impacts from one side the fertility but on the other side the animal behaviors such as sexual and marking behavioral attitudes. Indeed, as shown in the example 13, deslorelin administration impacts the penile spines and behaviors records as presented in FIGS. 14C and 14D and shows that for penile spines the results are statistically significant from 7 weeks until 67 weeks post injection. Regarding the Sexual Behaviors, the results were statistically significant 10, 12, 34, 36, 38, 46, 52, 58 and 66 weeks post-injection. Finally, with regard to the Reproductive behaviors, it was noted that there was no statistical difference between the two groups. The Libido was impacted by individual personality traits of cats. It was also noted a trend towards a decrease in vocalization, aggression, urine marking and typical male urine odor.

The present invention provides several advantages that include but are not limited to the following:

First of all, with an action upon two synthesis pathways of urine or feces odor, it is clear that the results in terms of odor attenuation, will be at least the addition of these two pathways results and certainly better than action only based on urine/feces odor synthesis mediated by sexual hormone pathway alone.

In comparison with surgically neutered animal, it is now shown in chemically neutered animal by deslorelin administration that the molecules responsible of urine or feces odor are quantitatively and qualitatively attenuated or reduced.

In addition, through the present disclosure it is demonstrated that the method of use deslorelin as described herein is a reliable, reversible, non-surgical neutering solution to foster pet family wellbeing while keeping options open.

The action on Testosterone pathway and particularly in reducing its blood level may contribute to the prevention of renal disease in said animal because it is known these kinds of disease are related with the accumulation of molecules such as trimethylamine.

It should be also mentioned that further to a safety study in cat, deslorelin administration at high dose reveals neither serious adverse events nor live threatening events. In addition, according to several tolerance studies in male and female species, it has been concluded that deslorelin, particularly administered as an implant (i-e Suprelorin®) was well tolerated.

Another object of the invention is the use of deslorelin for inducing temporary infertility in intact female animal and preferably in prepubere animal such as female cat or female dog over a period of at least 6 months, comprising administering deslorelin subcutaneously or intramuscularly to an animal in need thereof. Indeed as shown in example 14, deslorelin is effectively administered to prepubere female dog to delay puberty onset by using at least one deslorelin implant.

### EXAMPLES

### Example 1: Determination of Deslorelin Plasma Concentration of Over Time in Cat

The aim of the study was to determine the plasma concentration of deslorelin over time in the plasma of intact male cats after a single subcutaneous (SC) administration of the Suprelorin® 4.7 mg implant comprising as excipients hydrogenated palm oil, lecithin and sodium acetate anhydrous.

### 1.1 Materials and Methods:

study period: D0-560 (18 months)·
n=20 adults, sexually mature intact male cats
blinded, randomized
Suprelorin® group: n=16=>4.7 mg Suprelorin® SC, neck
Control group: n=4=>0.5 ml saline SC, neck

Blood samples were collected from 16 healthy, intact male cats that had received a deslorelin implant. Samples were analyzed using a validated liquid chromatography coupled to tandem mass spectrometry (LC-MS/MS) method after solid extraction using Oasis® WCX uElution plate. The calibration range was 4 pg/mL+2000 pg/mL. Concentrations measured below the lower limit of quantification (LLOQ) of 4 pg/mL were reported as BLQ (Below the Limit of Quantification).

Non-compartmental pharmacokinetic analysis of individual deslorelin concentrations in feline plasma was performed using Phoenix® software (WinNonlin 8.0, NLME 1.6, Certara L. P., Pharsight, St. Louis, MO, USA) to estimate the pharmacokinetic parameters of deslorelin.

### 1.2 Results:

A set of data showing Plasma deslorelin concentration (pg/ml) measured over time in Suprelorin® treated cats is presented in table 1 below:

TABLE 1

| | Minimum | Median | Average | Maximum |
|---|---|---|---|---|
| Days | conc. | conc. | conc. | conc. |
| 0.07 | 35.825 | 111.546 | 107.246 | 169.227 |
| 84 | 155 | 1.089 | 1.061 | 1.981 |
| 168 | 64 | 419 | 413 | 678 |
| 252 | 19 | 66 | 122 | 438 |
| 336 | 0 | 9 | 54 | 265 |
| 420 | 0 | 0 | 9 | 99 |
| 504 | 0 | 0 | 2 | 12 |
| 560 | 0 | 0 | 1 | 7 |

From this example it was determined that deslorelin is present in the blood in very high concentrations at DO and then declines continuously over time. Deslorelin remains present in the blood for up to 560 days in some cats.

### Example 2: Determination of Plasma Concentration of Testosterone in Cat

The aim of this study was to determine the plasma concentration of testosterone to monitor the efficacy of the deslorelin implant.

2.1 Materials and methods:

Samples were collected from 16 healthy, intact male cats that had received a deslorelin implant (Suprelorin® 4.7 mg). A Control Group of 4 healthy, intact male cats received saline solution. Testosterone was analyzed using a well-established and validated radioimmunoassay (RIA) as described previously (Basal testosterone concentrations after the application of a slow-release GnRH agonist implant are associated with a loss of response to buserelin, a short-term GnRH agonist, in the tom cat, Goericke-Pesch et al., Theriogenology. 2013 Jul. 1; 80 (1): 65-9). The lower limit of detection was 0.05 ng/ml. The intra- and inter-assay coefficients were 3.7 and 7.6%, respectively.

### 2.2 Results:

The results are shown in FIG. 1 on which the geometric means of the Testosterone Ratio Post/Pre GnRH stimulation and its 95% confidence interval (95% CI) is expressed as a function of time (in days).

At baseline, ratios Post/Pre GnRH stimulation were 9.39 for the Control group and 5.65 for the Treated Group. There was no significant difference between the two groups. The Ratio Treated/Control and its 95% Confidence Interval was 0.60 [0.18; 1.96] and p=0.3785.

As soon as Day 14, the ratio Post/Pre GnRH stimulation was significantly higher in the Control group, ratio Treated/Control was equal to 0.07 (p<0.0001), these differences lasted till Day 252, because on Day 280 the ratio Treated/Control was 1.37 and p=0.7004. On the next time points from Day 308 to Day 364 the superiority of the control group was established again (p<0.01). On Day 392 and onwards no statistically significant differences were observed.

Conclusion: This example shows that the testosterone concentration increases during the deslorelin implant (flare-up phase) and then decreases from the second week until day 252. From this day onwards, the testosterone concentration slowly increases and returns to a physiological concentration.

### Example 3: Deslorelin Influence on Sperm Concentration in Cat Male

The objective of this study was to investigate whether deslorelin has an influence on the sperm concentration of male cats.

3.1 Materials and method:

The test was performed on the Treated group and on the Control Group of the male cats of example 2 (see point 2.1 of example 2).

1 μL of semen was placed in 199 UL of citrate formalin to count the sperm concentration in the sample and perform an assessment of sperm morphology-when sufficient volume is available. The sperm concentration was determined by a the standard operating procedure. The dilution ratio was 1:200.

### 3.2 Results:

The results are shown in Table 2 below and in FIG. 2 in which the sperm concentration (/μL) and its interquartile range is expressed as a function of time (in days).

Despite an observed lower sperm concentration after Day 56 in the treated group, with median reaching 0 from Day 112 to Day 364, the distributions of the changes from baseline between the two treatments were not statistically significant except on Day 252 (p=0.0262). The Sperm concentration (/μL) is measured over time in treated cats (Suprelorin®). The details are present in table 2 below.

TABLE 2

| | Minimum | Median | | Maximum |
|---|---|---|---|---|
| Days | conc. | conc. | Average conc. | conc. |
| Pre- | 25 000 | 1 465 000 | 113 248.9 ± | 2 975 000 |
| treatment | | | 981039.70 | |
| 14 | 0 | 1 140 000 | 1 622 806.4 ± | 5 165 000 |
| | | | 1579194.5 | |
| 168 | 0 | 0 | 4 364.1 ± | 345 313 |
| | | | 122 086.58 | |
| 252 | 0 | 0 | 2 070.5 ± | 164 062.5 |
| | | | 53 518.08 | |
| 336 | 0 | 0 | 189 484.4 ± | 1 420 000 |
| | | | 445 581.92 | |
| 420 | 0 | 1 020 702 | 1 344 005.9 ± | 4 225 000 |
| | | | 1 297 420.4 | |
| 504 | 0 | 256 250 | 814 710.8 ± | 3 610 000 |
| | | | 1 128 723.9 | |
| 560 | 0 | 267 190 | 754 072.0 ± | 3 000 000 |
| | | | 943 046.19 | |

### Example 4: Determination of Direct Correlation Between Injected Deslorelin Concentration and Testosterone Concentration

The objective of this study was to investigate whether there is a direct correlation between injected deslorelin concentration and testosterone concentration.

### 4.1 Materials and Methods:

The apparent relationship between plasma deslorelin concentrations and PD parameters over time was assessed graphically from the testosterone and deslorelin concentrations obtained in 16 healthy male cats treated with Suprelorin® 4.7 mg.

The serum concentration of testosterone was measured according to the protocol detailed in example 2, point 2.1.

The plasmatic concentration of deslorelin was measured according to the protocol detailed in example 1, point 1.1.

### 4.2 Results:

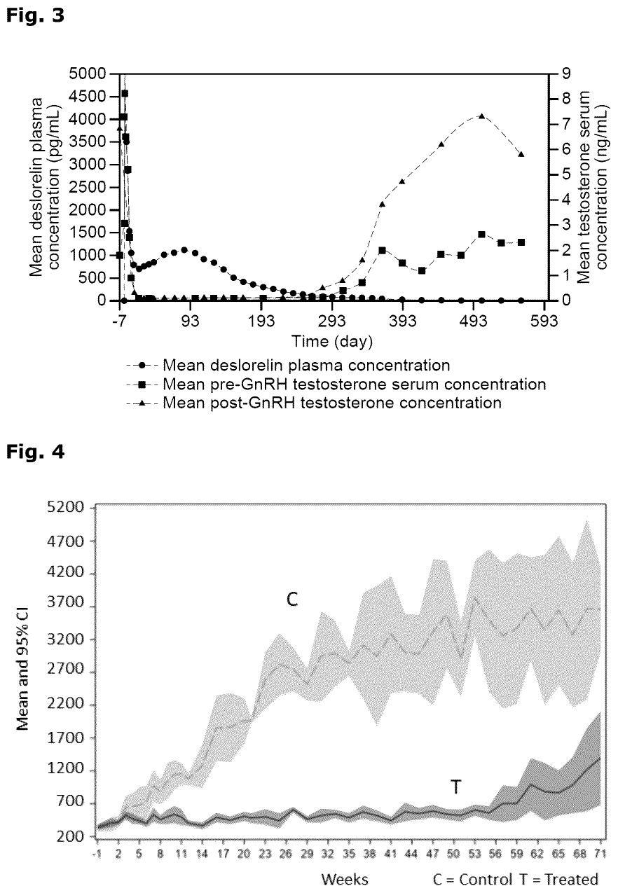

The results of mean deslorelin plasma concentration and the mean Testosterone (pre- and post-GnRH stimulation) serum concentration over time (n=16 treated cats) are shown in FIG. 3. On this figure, mean deslorelin plasma concentration (black full circles) (in pg/mL), mean pre-GnRH testosterone serum concentration (black full squares) (in pg/mL), and mean post-GnRH testosterone serum concentration (black full triangles) (in pg/mL) are expressed as a function of time (days).

Conclusion: This study showed that there was an inverse correlation between deslorelin and testosterone. Indeed, as soon as the concentration of deslorelin became low (inferior at 12 pg/mL), the concentration of testosterone started to increase.

### Example 5: Determination of Plasma Concentration of Deslorelin Over Time and to Validate an LC-MS/MS Method

The objective was to determine the plasma concentration of deslorelin over time, and to validate an LC-MS/MS method for the determination of deslorelin in the plasma of intact male cats after a single subcutaneous administration of the Suprelorin® 4.7 implant.

### 5.1 Materials and Methods:

Samples were collected from 10 intact male cats treated with Suprelorin® 4.7 implant. Samples (based on a plasma volume of 150 μL, treated with K2-EDTA as anticoagulant) were analyzed by a LC-MS/MS method after a solid extraction phase using μElution Oasis® WCX plates. The compound (13C, 15N)-Leu (7)-deslorelin was used as a standard. The calibration range was 4 pg/mL-2000 pg/mL.

### 5.2 Results:

The results of measurements of plasma concentration of deslorelin over time in cats treated with a deslorelin implant (i-e Suprelorin® 4.7 mg are presented in table 3 below.

TABLE 3

| Day(s) | 0.0833 | 84 | 168 | 252 | 336 | 420 | 497 |
|---|---|---|---|---|---|---|---|
| Minimum | 39,862 | 159 | 77 | 31 | 15 | 0 | 0 |
| conc. | | | | | | | |
| Median | 95,877 | 626 | 379 | 192 | 70 | 14 | 3 |
| conc. | | | | | | | |
| Average | 103,333 | 624 | 355 | 213 | 78 | 16 | 12 |
| conc. | | | | | | | |
| Maximum | 227,126 | 1302 | 669 | 381 | 209 | 61 | 75 |
| conc. | | | | | | | |

Conclusion: This example has shown that deslorelin is present in the blood in very high concentrations at DO and then there is a decline in this concentration over time. Deslorelin remains in the blood for up to 497 days in some cats.

### Example 6: Determination of Serum Testosterone Concentrations Using a Radioimmunoassay (RIA) Method

### 6.1 Objective:

The Serum testosterone concentrations after a single SC administration of the Suprelorin® 4.7 implant to intact male cats were collected to provide information on the effect of the implant on baseline testosterone levels and on the ability to produce testosterone after stimulation.

### 6.2 Materials and Methods:

Samples were collected from 10 intact male cats treated with Suprelorin®. Samples were analyzed using a radioimmunoassay (RIA) method with a validated extraction and a LLOQ of 0.01 ng/ml. Samples were analyzed in duplicate, with 350 UL allowing for duplicate analysis. Only results between 0.01 ng/ml and 2 ng/ml and with a coefficient of variation (CV %) less than or equal to 20% are reported.

### 6.3 Results:

The results of measurements of serum testosterone concentrations after a single SC administration of the Suprelorin® 4.7 implant to intact male cats over time are presented in table 4 below.

TABLE 4

| Day(s) | 5 | 84 | 140 | 280 | 336 | 364 | 420 | 497 |
|---|---|---|---|---|---|---|---|---|
| Minimum | 0.02 | 0.019 | 0.02 | 0.01 | 0.01 | 0.01 | 0.01 | 0.02 |
| conc. (ng/mL) | | | | | | | | |
| Median con. | 0.06 | 0.02 | 0.02 | 0.02 | 0.02 | 0.01 | 0.03 | 2.15 |
| (ng/ml) | | | | | | | | |
| Average | 0.12 | 0.02 | 0.02 | 0.02 | 0.02 | 0.11 | 0.72 | 1.59 |
| conc. (ng/mL) | | | | | | | | |
| Maximum | 0.49 | 0.025 | 0.03 | 0.02 | 0.02 | 1.02 | 2.73 | 3.79 |
| conc. (ng/mL) | | | | | | | | |

### Example 7: Deslorelin Influence on Testicular Size

### 7.1 Objective:

The objective was to see if deslorelin had an influence on testicular size

### 7.2 Materials and Methods:

In order to carry out this study, two groups of cats were set up:
Control group (5 cats, intact male cats)
Group treated by Suprelorin® 4.7 mg (10 cats, intact male cats)

The size of testicles of male cats (length, width and depth of each testicle in mm) were measured using calipers and recorded as a rounded number to the tenth of a mm.

### 7.3 Results:

The results of mean testicular volume over time curve (n=5 control cats and n=10 treated cats) are shown in FIG. 4.

At baseline, mean testicular volumes were similar between treatments with 323.5 and 340.8 mm³ for Control and Treated cats respectively, and median values were equal to 318 and 324.5 mm³. Overtime, mean values increased dramatically in Control cats to reach 3661.5 mm³ at Week 71/Month 18 (an increase of ×10 from baseline) whereas mean values fluctuated smoothly for the Treated group with a final value of 1394.8 mm³ (an increase of ×3 from baseline).

As early as Week 4/Month 1, the mean testicular volumes differed significantly from each other with a mean value (mm³) and 95% CI of 664.0 [465.7; 862.3] for Control cats and 466.4 [388.3; 544.5] for Treated cats. The observed difference between Treated and Control cats and the 95% CI was-197.3 mm³ [−347.7; −47.5]. This difference between treatments remained significant (−2266.7 mm³ [−3313.7; −1219.6]) until the end of the study, with a mean value (mm³) and 95% CI of 3661.5 [3006.4; 4316.4] for Control cats and 1394.8 [676.8; 2112.7] for Treated cats at Week 71/Month 18. When compared to testosterone post/pre GnRH stimulation ratio for which no more significant differences between groups was shown from D364/Week 53 (one calendar year), this shows that reversibility of effect for testicular volume occurs later.

Conclusion: This study showed that deslorelin induced a decrease in testicular volume in treated cats.

### Example 8: Analysis of the Pharmacokinetic Parameters of Deslorelin in Dogs

The aim of this study was a descriptive analysis of the pharmacokinetic parameters of deslorelin in dogs

### 8.1 Materials and Methods:

This study was carried out in 6 intact male dogs implanted with Suprelorin® (4.7 mg), from which blood was collected for the duration of the study (approximately 15 months). The objective of this bioanalytical study was to validate LC-MS/MS method for the determination of deslorelin in canine plasma (based on a plasma volume of 150 μL, K2-EDTA as anticoagulant). The lower LOQ (LLOQ) was established at 4 pg/mL in canine plasma.

### 8.2 Results:

The results of plasma concentration of deslorelin (pg/ml) measured over time in dogs treated with Suprelorin® is presented in table 5 below.

TABLE 5

| Days | Average deslorelin concentration (pg/ml) |
|---|---|
| 0 | 0 |
| 0.04 (1 hour) | 21,684 |
| 1 | 1,005 |
| 10 | 354 |
| 57 | 165 |
| 112 | 89 |
| 196 | 47.9 |
| 210 | 16 |
| 308 | 5 |
| 414 | 3 |
| 441 | 2 |

Conclusion: This example determined that deslorelin is present in the blood in very high concentrations at within the first hours of administration and then there is a decline in this concentration over time. Plasma deslorelin concentrations were quantifiable until the end of the study (D441) in some dogs with an average concentration of 2 pg/mL.

### Example 9: Deslorelin Influence on Testosterone Concentration

### 9.1 Objective:

The objective of this study was to see if the deslorelin implant had an effect on testosterone concentration.

### 9.2 Materials and Methods:

This study was conducted in 6 intact male dogs implanted with Suprelorin® (4.7 mg), from which blood was collected for the duration of the study (approximately 15 months).

Prior to testing, testosterone was extracted from serum. Serum samples were thawed at room temperature, vortexed and processed for steroid extraction. Briefly, 500 μL of serum was transferred to a clean glass tube and 2.5 mL of ethyl acetate was added. The mixture was vortexed for 3 min, and after allowing the layers to separate, the upper phase was transferred to clean test tubes. This procedure was repeated twice. The combined extracts were then evaporated by heating to 30° C. under a gentle stream of compressed air. The dried extracts were stored at −20° C. for 18 hours before analysis.

The Cayman Chemical Testosterone ELISA kit (ref 582701, Ann Arbor, USA) was used to measure testosterone levels in canine plasma.

### 9.3 Results:

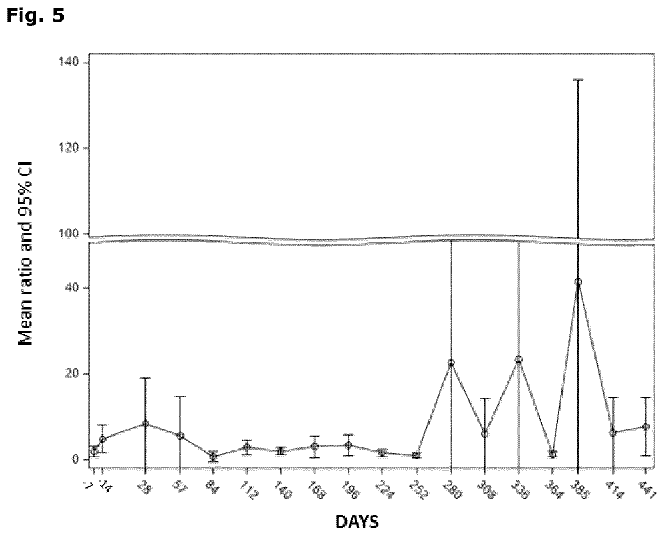

The results of mean ratios (and its 95% CI) of post/pre GnRH stimulation testosterone in treated dogs (n=6), measured over approximately 16 months of study are presented in FIG. 5.

Conclusion: This study demonstrates that upon injection of the implant (DO) there is an initial stimulation phase (DO to D28) of testosterone followed by an inhibition phase from 28 days to 252 days. Thus the injection of the implant induces a long-term decrease in testosterone.

### Example 10: Deslorelin Influence on Lh Concentration

### 10.1 Materials and Methods:

This study was conducted in 6 intact male dogs implanted with Suprelorin® (4.7 mg), from which blood was collected for the duration of the study (approximately 15 months).

LH was measured with the ELISA kit “LH Detect®” (ReproPharm Vet, France). Serum was diluted 1:5 and incubated with the coated capture antibody. After washing, a second antibody was incubated. After washing, a third conjugated antibody was finally incubated. After washing again, detection of bound conjugated antibody was performed by using tetramethyl benzidine (TMB) as the substrate. Reaction was stopped by acidic solution. The absorbance was read at 450 nm and samples absorbance values were compared with calibration curve values (0-8 ng/ml) to determine samples concentrations. LH values were expressed in ng/ml.

### 10.2 Result:

The results of mean ratios (95% CI) post/pre-GnRH stimulation LH ratios in treated dogs (n=6) measured for approximately 15 study months are presented in FIG. 6.

At baseline, mean post/pre-GnRH stimulation ratio was 15.8. Between D28 and D168, mean ratios were equal to 1. On D252, this ratio started to increase to reach a mean of 5.8. From there and up to the end of the study, mean ratio values were around 10.

From D28 to D168, all dogs had a ratio below 2. On D196 and D224, only one dog had a ratio greater or equal to 2. After D252, all dogs had ratio values≥2.

### Example 11: Correlation Between the Concentration of Injected Deslorelin and the Concentration of the Hormones LH, FSH And Testosterone in Male Cats Treated with Suprelorin®

### 11.1 Objective:

The objective of this study was to investigate whether there was a direct correlation between the concentration of injected deslorelin and the concentration of the hormones LH, FSH and testosterone in male cats treated with Suprelorin® (4.7 mg).

### 11.2 Materials and Methods:

This study was performed in 6 intact male dogs implanted with Suprelorin® (4.7 mg), from which blood was collected for the duration of the study (approximately 15 months). The apparent relationship between plasma deslorelin concentrations and PD parameters over time was assessed graphically from the testosterone/LH/FSH and deslorelin concentrations obtained in the 6 healthy male cats treated with Suprelorin® 4.7 mg.

### 11.3 Results:

The results are reported on FIG. 7.

FIG. 7 shows the initial spike followed by a drastic decrease in mean LH concentration followed by testosterone concentrations (slight for FSH) and the gradual decrease in deslorelin plasma concentrations versus pre-GnRH testosterone, LH and FSH concentrations over time which intercepts with an increase of all hormone levels on D224 when deslorelin concentration was at 12±10.4 pg/mL.

Conclusion: This study showed that there was an inverse correlation between deslorelin and testosterone/LH and FSH. Indeed, as soon as the deslorelin concentration became low (inferior at 12 pg/mL), the other concentrations started to increase.

### Example 12: Cat Field Studies

The aim of this studies was to demonstrate the suppression of fertility and persistence of infertility in cat male animals.

### 12.1. Materials and Methods

This example provides the data collected from 2 combined studies (Double-masked Placebo-Controlled randomized) that have been conducted sequentially according to the following schedule:
Study 1: The study 1 has been conducted on a group of 205 male cats divided into two groups, one Control Group that received 1 mL of saline solution (n=51-subcutaneous administration) and one Treated Group (n=154) that received an implant of Suprelorin® 4.7 mg (subcutaneous implant-deslorelin acetate).
Study 2: At the end of a first period of 12 months, the Suprelorin® implant was removed in all the male cats belonging to the Treated Group. 12 male cats of the Treated Group issued from study 1 were then administered saline solution (1 mL subcutaneously) (Saline Goup) and 22 cats of the Treated Group issued from Study 1 were re-implanted with a new Suprelorin® 4.7 mg implant for a second period of time of 12 months (Treated twice Group). The efficacy and safety of the re-implantation was followed-up for these supplementary 12 months, i.e. for a total of 24 months.

The observation schedule was as follows:
Study 1: V1: Day −14 to −7; V2: Day 0; V3: 1.5 months±3 days; V4: 3 months±5 days, V5: 6 months±5 days, V6: 9 months±5 days; V7: 12 months #5 days;
Study 2: V8: 12 months±3 days; V9: 13.5 months±3 days; V10: 15 months±5 days; V11: 18 months±5 days; V12: 21 months±5 days; V13: 24 months±5 days.

Regarding the enrolment criteria, in both studies the criteria were as the following:
Domestic Cats
Age >3 MONTHS old or >15 MONTHS in Study 2
Intact Indoor males
Client-owned (including shelter cats/rehomed by foster families)

With the additional criteria for study 2 of being a success in study 1.

In these studies the following primary endpoints were measured:
For Study 1: the suppression of fertility (Testosterone≤0.10 ng/ml* from V4 until V7*-3 to 12 months)
Study 2: the suppression of fertility (Testosterone≤0.10 ng/mL*from V8 until V13 (12 to 24 months) and the time to reversion effect.

In addition to the safety parameters (Haematology and blood biochemistry, Urinalysis, Local reactions at the implantation site, Change in Body weight, Appetite, Abnormal testicular evaluation, Adverse events), the following secondary endpoints have been reported as well:
Sexual behaviour sum score
Aggression
Vocalization
Urine marking
Urine Smell/odour
Penile Spines
Testicular volume

### 12.2 Results:

### —Testosterone:

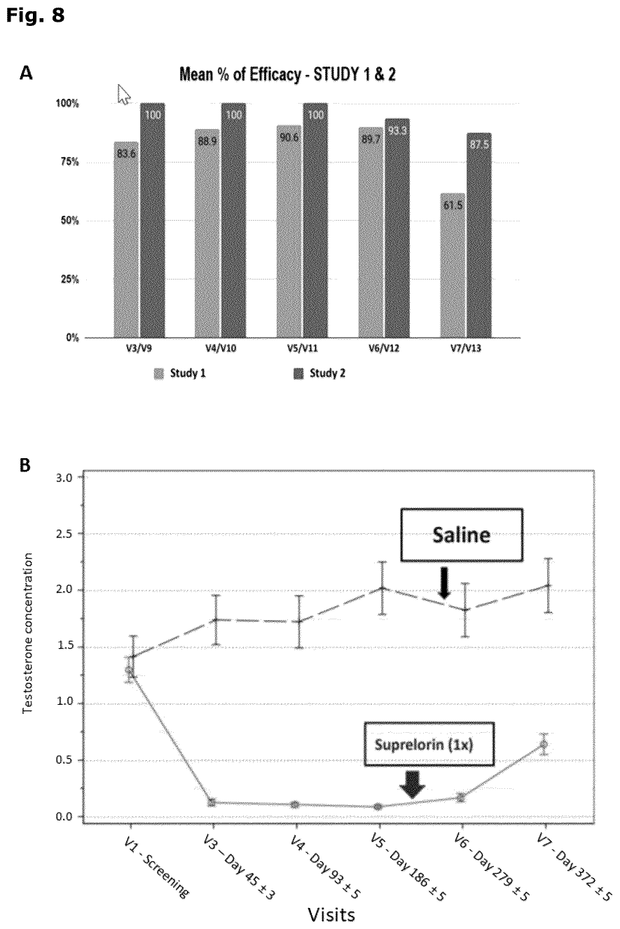

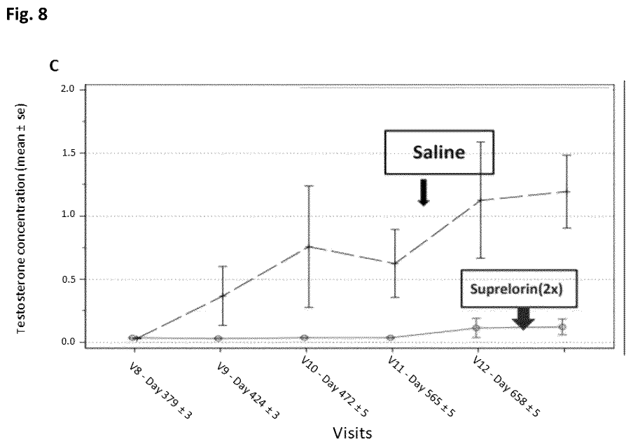

The mean of percentages of efficacy results are presented in FIG. 8A. Overall, the success rate of both studies was very similar for 1 or 2 implants with an Efficacy >87% at all time points in Study 2.

Detailed levels of Testosterone are respectively presented in FIG. 8B for study 1 and in FIG. 8C for study 2.

### —The Reversibility:

The reversibility of deslorelin effect has been studied. Indeed, among the deslorelin Treated Group of study 1, n=12 subject were enrolled in the Saline group for study 2 as explained above in point 12.1.

The results showed that 100% of the cats issued from the Treated Group of Study 1 and enrolled in the Saline Group of Study 2 reversed at D279 after being enrolled in Study 2. Median time for reversibility based on serum testosterone concentration measurement was D379±178.

### —the Secondary Endpoints:

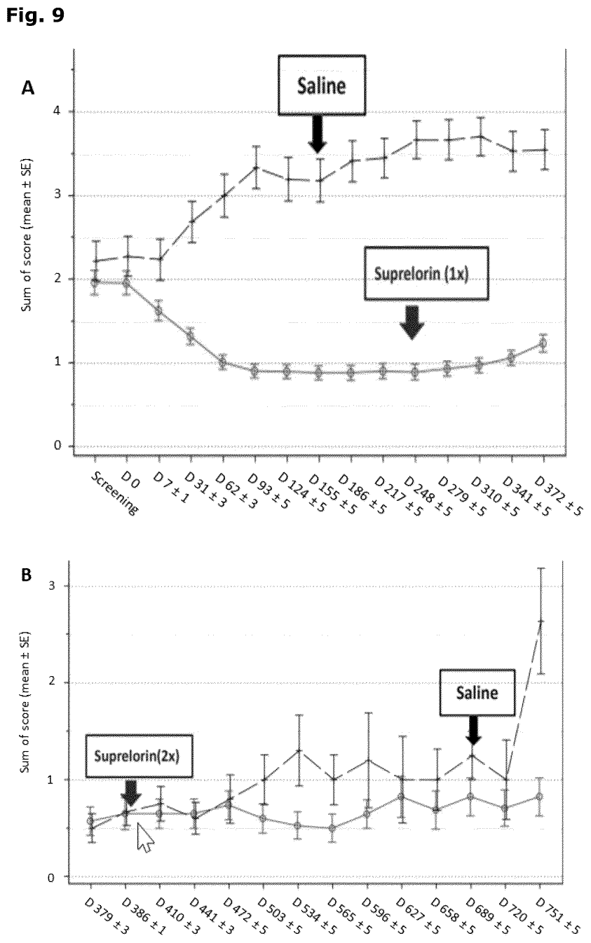

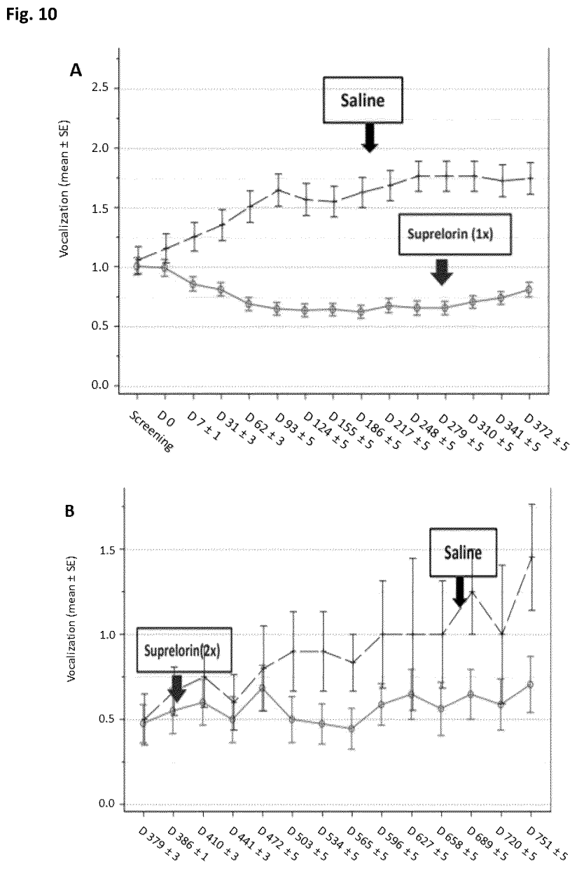

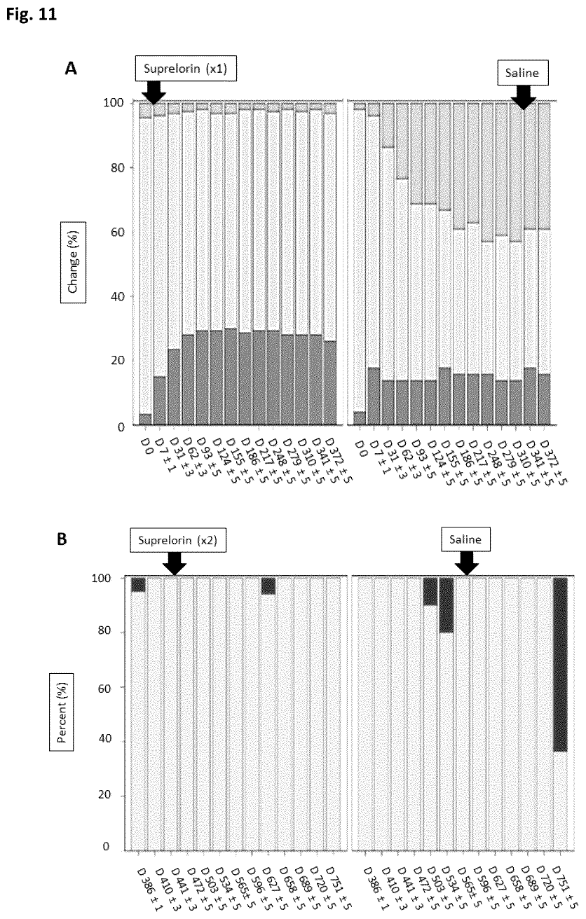

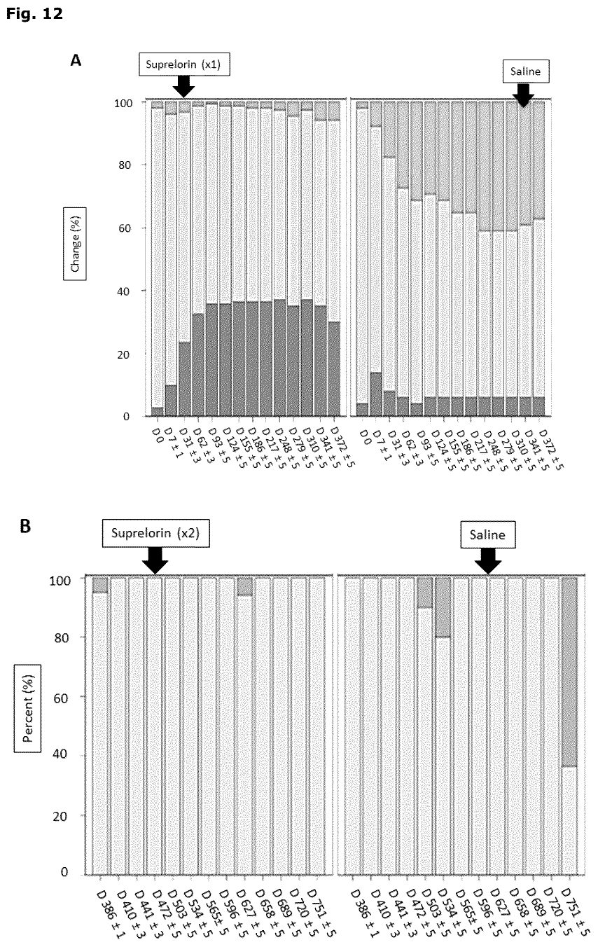

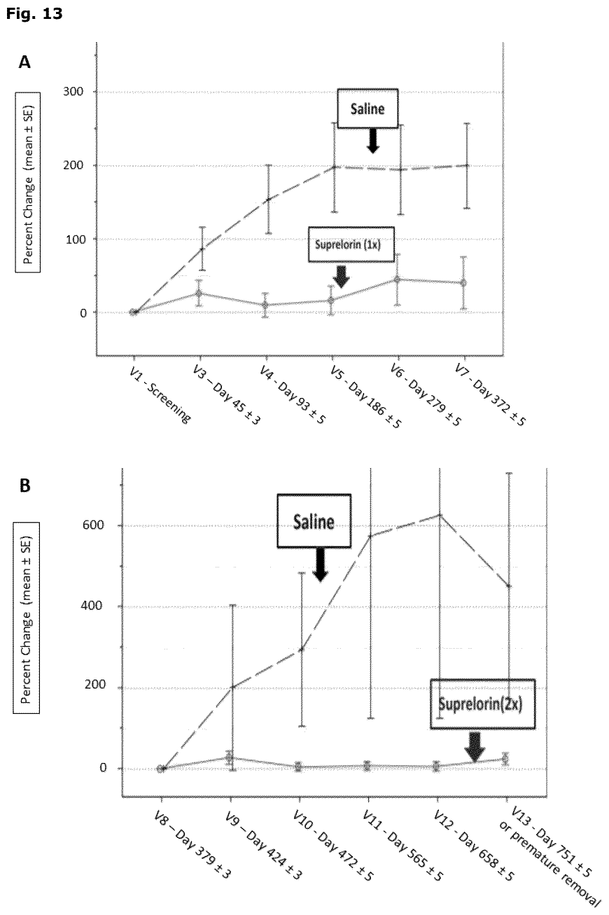

The efficacy results of the secondary endpoints have been reported with:
the mean of sum score of sexual behaviors respectively presented in FIGS. 9A for study 1 and 9B for study 2,
the mean vocalization variation in FIGS. 10A for study 1 and 10B for study 2,
the Urine marking, the % change from baseline is respectively presented for study 1 in FIGS. 11A and for study 2 in FIG. 11B,
the urine odor variation/reduction measured as the change from baseline are presented respectively for study 1 in FIGS. 12A and for study 2 in FIG. 12B
the % change of testicular volume is presented respectively for study 1 in FIGS. 13A and for study 2 in FIG. 13B
the % change of appearance penile spines is presented respectively for study 1 in FIGS. 14A and for study 2 in FIG. 14B

During the course of the studies, there were no safety concerns and the deslorelin implants were well tolerated.

In conclusion, these combined studies have demonstrated that:

The deslorelin was effective for at least 12 months in chemically castrating the subjects. This effect has been proved to be reversible at the end of the treatment course of action. These studies have also demonstrated that sexual behaviors have been reduced with a quick onset of efficacy in behavioral reduction (approx 7-31 days). In addition there has been no safety concern either with the single administration or with the repeated administration.

Therefore, it can be concluded that deslorelin administered with a slow release and particularly with an implant is a good alternative to the surgical castration and is a safe non-surgical method for controlling male cat reproduction.

The method of use deslorelin as described herein is a reliable, reversible, non-surgical neutering solution to foster pet family wellbeing while keeping options open.

### Example 13: PK/PD Preclinical Study in Intact Male Cats (Intact Male Cats)

The aim of this studies was to demonstrate the suppression of fertility and measure the deslorelin levels with the PK data, evaluate the reversibility with PD analysis and record the systemic and local tolerance.

This example provides the data collected from PK/PD preclinical study in intact male cats according to the following study design:
Product: Suprelorin® 4.7 mg vs. placebo (NaCl solution)
Subject: Cats treated at 15 weeks of age=Fifteen+3 intact male cats (15 weeks old) were subdivided in two groups; one group treated with Suprelorin® 4.7 mg implant (n=13-Group 2) and the other group treated with saline solution (placebo) (n=5, Group 1). On D14, the three spare cats were removed from the study. Five+3 female cats (Group 3), pubescent at study start, were included in the study solely to assess the reproductive behavior of male cats. They were not treated with the implant.
Duration: 18 Study Months post-injection (PI) (D502/Week 72, i.e., 16.5 calendar months)

Table 6 below gives the details of the study:

TABLE 6

| | Group 1 | | |
|---|---|---|---|
| | Control | Group 2 | Group 3 |
| Activity | cats | Treated male cats | Female cats |
| Treatment | NaCl | Suprelorin ® 4,7 mg | Not treated |
| Animals | 5 male | 10 + 3 male cats | 5 + 3 Females |
| | cats | | |
| Clinical exam and | X | X | X |
| observation (BW) | | | |
| Local tolerance | X | X | — |
| (Implantation) | | | |
| Heat monitoring | — | — | X |
| Plasma deslorelin | — | X | — |
| analysis PK | | | |
| LC-MS/MS + | | | |
| WinNonLin ® | | | |
| Serum | X | X | — |
| testosterone | | | |
| analysis | | | |
| RIA: pre & post- | | | |
| GnRH stim + ratio | | | |
| Testicular volume | X | X | — |
| & penile spines | | | |
| Sexual behavior | X | X | — |
| assessment | | | |
| (aggression, | | | |
| vocalization, urine | | | |
| marking and | | | |
| odor) | | | |
| Reproductive | X | X | X |
| behavior | | | |
| assessment | | | |
| (Libido, mounting, | | | |
| mating) | | | |

In this table, BW means Body Weight.

### Study Results:

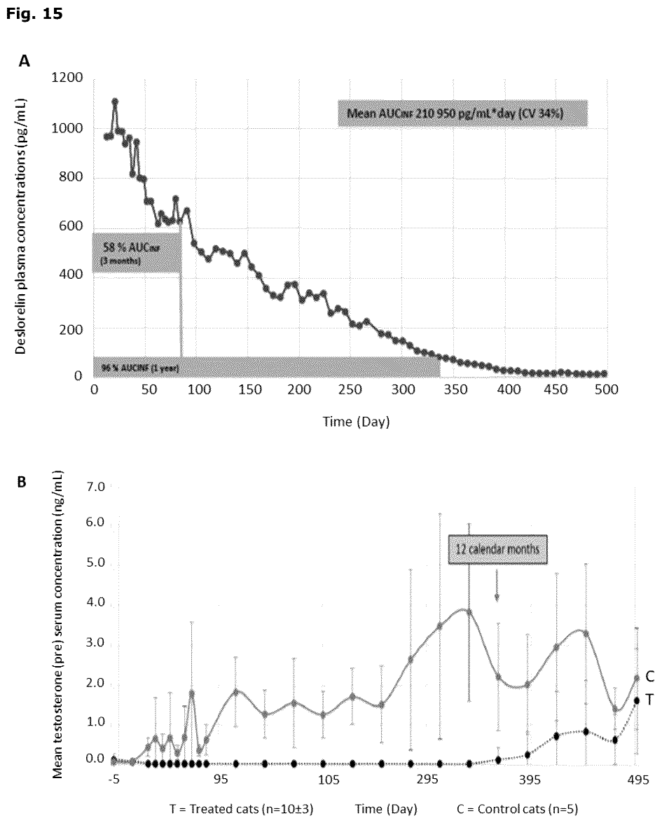

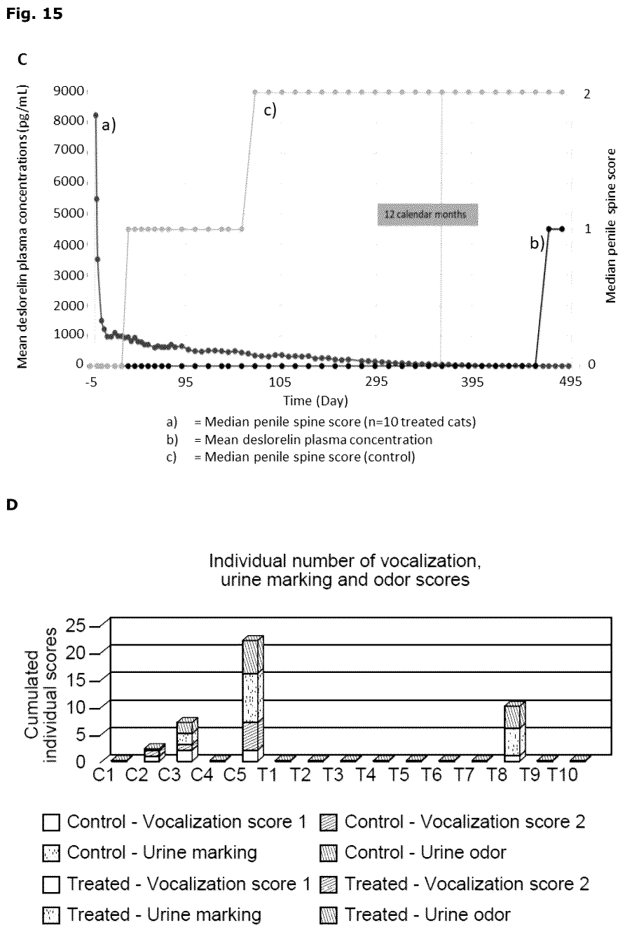

The PK_study results are shown in FIG. 15A. Overall, it should be noted that the Tmax is at 2 hours, decrease of mean 92% in the first 24 h post-injection (PI) then steady and slow decrease. The first cat with undetected concentration at 1 year PI (52 weeks PI) and 5/10 male at the end of the study.
The levels records of testosterone are presented in FIG. 15B and show that the testosterone is low in Treated cats until one year post PI (53 weeks PI), then increased (except 3/10 cats). At the end of the study, the mean testosterone is still lower in Treated cats (Pre-GnRH: 1.59 vs 2.16 ng/ml/Post-GnRH: 2.34 vs. 6.44 ng/ml in Treated/Controls, respectively).

It is noticeable that post/pre ratio were statistically significant from 2 weeks until 1 year PI (p=0.0001)
The Penile spines and behaviors records are presented in FIGS. 15C and 15D and show that for penile spines the results are statistically significant from 7 weeks until 67 weeks post injection. Regarding the Sexual Behaviors, the results were statistically significant 10, 12, 34, 36, 38, 46, 52, 58 and 66 weeks post-injection. Finally, with regard to the Reproductive behaviors, it was noted that there was no statistical difference, one control male mated 35 weeks post-injection (at 1 year old) and one treated cat mated 65 weeks post-injection. The Libido was impacted by individual personality traits of cats. It was also noted a trend towards a decrease in vocalization, aggression, urine marking and typical male urine odor as shown in FIG. 15D. It is clear from said figure that cats from the control group (animals C1 to C5) present more sexual activity or reproductive behaviors including vocalization, aggression, urine marking and typical male urine odor compared to the treated animals (T1 to T10) with the deslorelin implant.
The puberty appeared at 5.5 months of age in the 5 intact control cats. The puberty onset was delayed in the deslorelin treated cats and lasted at least 1 year.

The Complete reversal of andrological and hormonal parameters was effective in 5/10 Treated cats at the end of the study (72 weeks PI).

### Example 14: Use of Deslorelin in Prepubertal Female Dogs

The aim of this study was to evaluate the delay of puberty onset in prepubertal female dogs. As known the puberty begins with the setting up of several parameters:
Increasing number of receptors in central nervous system (CNS) and gonads
Desensitization of hypothalamus/pituitary towards estrogens
Ripening of the gonads and the secondary sexual organs
Follicle ripening, spermatogenesis

The results of this study show that the delay of puberty is possible:

With 1 implant: there was no disturbance of body development, the vulva size was normal, but delayed epiphyseal closure. A Slight flare-up possible (vulva swelling)

With 3 implants every 4.5 Mo: it was noticed that vulva stayed juvenile and the effect was comparable to prepubertal castration.

For notice, the concentration of deslorelin not related to duration of efficacy and the Implant must not be removed.

This study allows to determine the criteria of deslorelin administration to prepubertal female dogs:
Age at implant insertion should be 4-5 months of age.
the beginning puberty must be excluded where determination is done by clinical examination, vaginal inspection, vaginal cytology, E2, DHEA measurement (sexual behavior)

1-12. (canceled)

13. A method for chemically castrating a non-human mammal within 1 month after administration, wherein said method comprises a step of administering an effective amount of deslorelin so as to obtain a Cmax concentration of deslorelin that exceeds 7000 pg/mL and a deslorelin concentration in the non-human mammal plasma that is at least 10 pg/mL.

14. The method according to claim 13, wherein the deslorelin is administered subcutaneously or intramuscularly to a non-human mammal in need thereof, at a dosage efficient to induce a deslorelin plasma concentration between 4000 pg/mL and 40 000 pg/mL between 1 h and 24 hours, and a plasma concentration below 400 pg/ml from 8 days after administration.

15. The method according to claim 14, wherein the deslorelin plasma concentration is between 10 pg/mL and 400 pg/ml from 8 days after administration up to at least 6 months.

16. The method according to claim 14, wherein the deslorelin concentration in the non-human mammal plasma is maintained for at least 6 months.

17. The method according to claim 14, wherein said method comprises the step of administering to said animal a sustained-release drug delivery system (DDS) containing deslorelin, and wherein the deslorelin concentration in the non-human mammal plasma is maintained for at least 6 months.

18. A method for inducing temporary infertility in intact male animal over a period of at least 6 month, comprising administering deslorelin subcutaneously or intramuscularly to a mammal in need thereof, at a dosage efficient to induce a deslorelin plasma concentration between 4000 pg/mL and 40 000 pg/mL between 1 h and 24 hours, and a plasma concentration below 400 pg/ml since 8 days after administration.

19. The method according to claim 18, wherein after administration, the deslorelin is released substantially continuously from 8 days after administration to at least 6 months at a level plasma concentration below 400 pg/mL.

20. A method for chemically castrating a non-human mammal, wherein said method comprises a step of administering to said animal a sustained-release drug delivery system (DDS) containing deslorelin, wherein said DDS releases deslorelin with a Cmax of deslorelin after administration that exceeds 7000 pg/mL and wherein a deslorelin concentration in the non-human mammal plasma is at least 10 pg/mL and maintained for at least 6 months.

21. The method according to claim 20, wherein said DDS is a subcutaneous or intramuscular implant comprising deslorelin and a biodegradable material.

22. A method for chemically castrating a non-human mammal, wherein said method comprises the step of administering an effective amount of deslorelin so as to obtain a decrease of the Testosterone levels in plasma of said non-human mammal below 0.4 ng/ml and a deslorelin concentration in the non-human mammal plasma that is at least 10 pg/mL.

23. A contraceptive method of a non-human mammal comprising administering subcutaneously or intramuscularly to said non-human mammal, an implant comprising deslorelin and a biodegradable material, wherein the implant releases deslorelin with a Cmax of deslorelin at 1 hour after administration exceeding 7000 pg/mL and wherein a deslorelin concentration in the non-human mammal plasma is at least 10 pg/mL and maintained for at least 6 months.

24. The contraceptive method according to claim 23, wherein the deslorelin is administered to a non-human mammal in need thereof, at a dosage efficient to induce a deslorelin plasma concentration between 4000 pg/mL and 40000 pg/mL between 1 h and 24 hours, and a plasma concentration below 400 pg/ml since 8 days after administration.

25. The contraceptive method according to claim 23, wherein the plasma concentration of deslorelin is below 400 pg/ml since 8 days after administration up to at least 6 months.

26. The contraceptive method according to claim 24, wherein the plasma concentration of deslorelin is below 400 pg/ml since 8 days after administration up to at least 6 months.
